(19) United States
(12) Patent Application Publication
(10) Pub. No.: US 2023/0323330 A1
(43) Pub. Date: Oct. 12, 2023

(54) POLYESTER DEGRADING PROTEASE VARIANTS

(71) Applicant: Novozymes A/S, Bagsvaerd (DK)

(72) Inventors: Rolf Thomas Lenhard, Lyngby (DK); Hanne Lyngby Hoest Pedersen, Stenlose (DK)

(73) Assignee: Novozymes A/S, Bagsvaerd (DK)

(21) Appl. No.: 18/043,515

(22) PCT Filed: Aug. 30, 2021
(86) PCT No.: PCT/EP2021/073917

§ 371 Date: Feb. 28, 2023

(30) Foreign Application Priority Data
Aug. 28, 2020 (EP) 20193301.7
Feb. 12, 2021 (EP) 21156744.1

(51) Int. Cl. C12N 9/54; C08J 3/20; C08K 11/00

(52) U.S. Cl. CPC C12N 9/54; C12Y 304/21062; C08J 3/201; C08K 11/00; C08J 2367/04

(57) ABSTRACT

The present invention relates to protease variants having polyester degrading activity, polynucleotides encoding said variants, nucleic acid constructs and expression vectors comprising said polynucleotides, host cells expressing said variants, compositions comprising the variants, methods of producing the variants, and use of the variants.

### REFERENCE TO A SEQUENCE LISTING

This application contains a Sequence Listing in computer readable form, which is incorporated herein by reference.

### FIELD OF THE INVENTION

The present invention relates to protease variants having polyester degrading activity, polynucleotides encoding said variants, nucleic acid constructs and expression vectors comprising said polynucleotides, host cells expressing said variants, compositions comprising the variants, methods of producing the variants, and use of the variants.

### BACKGROUND OF THE INVENTION

Proteases are able to catalyze the hydrolysis of a variety of polymers, including polyesters. In this context, proteases have shown promising effects in several industrial applications, including as detergents for dishwashing and laundry applications, as degrading enzymes for processing biomass and food, as biocatalysts in the detoxification of environmental pollutants, and for the treatment of polyester fabrics in the textile industry. Likewise, the use of proteases as enzymes for hydrolyzing polylactic acid (PLA) is of particular interest. Indeed, PLA is a bio-based polymer that is used in a large number of technical fields, such as flexible and rigid packaging, bags, mulching films, as well as in the manufacture of clothes and carpets. Accordingly, PLA accumulation in landfills becomes an increasing ecological problem.

Among proteases, serine proteases (EC 3.4.21) are enzymes that cleave peptide amide bonds in proteins, in which serine serves as the nucleophilic amino acid in the enzyme active site. Serine proteases are found ubiquitously in both eukaryotes and prokaryotes. Numerous bacterial serine proteases have been identified initially in Bacillus and more recently in other mesophilic hosts. However, an increasing number of serine proteases have been isolated from thermophilic and hyperthermophilic bacteria. As an example, aqualysin I, from Thermus aquaticus YT-I, has been cloned, sequenced and expressed in Escherichia coli.

Biological degradation, and more particularly enzymatic degradation, is considered as an interesting solution to decrease plastic waste accumulation. Indeed, enzymes are able to accelerate hydrolysis of polyester containing material, and more particularly of plastic products, even down to the monomer level. Furthermore, the hydrolysate (i.e., monomers and oligomers) can be recycled as material for the synthesis of new polymers. Recently, new plastic materials have been developed that integrate biological entities suitable for degrading at least one polymer of the plastic material, leading to the production of biodegradable plastic products. As an example, plastic products made of PLA and including proteases have been produced. Such biodegradable plastics may at least partially solve the problem of plastic build-up in landfill sites and natural habitats.

In this context, several proteases have been identified as candidate degrading enzymes. For instance, a protease of Micromonospora sp. (WO 2016/146540) has been described for its capacity to degrade polyester, and more particularly polylactic acid. Further examples of PLA-degrading proteases are described in WO 2018/109183 and WO 2019/122308.

However, there is still a need for proteases with improved activity and/or improved stability at high temperatures to allow a degradation process with higher efficiency, and thereby enhancing the competitiveness of biodegradable plastic production processes, biological polyester degradation processes and/or biological recycling processes.

### SUMMARY OF THE INVENTION

The present invention provides protease variants with improved polyester degrading activity and improved stability.

In a first aspect, the present invention relates to protease variant having a sequence identity of at least 60%, but less than 100%, to SEQ ID NO:1; wherein the variant comprises the substitutions X99F and X101L; wherein the variant further comprises at least one substitution selected from the group consisting of X3T, X97D, X103T, X104I, X127I, X161W, X161R, X161Y, X161L, X161V, X163R, X172K, X174G, X174V, X175A, X175Y, X175D, X175T, X175V, X175I, X176S, X194P, and X215K; wherein the variant has polyester degrading activity and, optionally, protease activity; and wherein position numbers are based on the numbering of SEQ ID NO:2.

In a second aspect, the present invention relates to an isolated polynucleotide encoding a variant of the first aspect.

In a third aspect, the present invention relates to a nucleic acid construct or expression vector comprising a polynucleotide of the second aspect, or a recombinant host cell transformed with a polynucleotide of the second aspect.

In a fourth aspect, the present invention relates to a composition comprising a protease variant according to the first aspect.

In a fifth aspect, the present invention relates to a method of producing a protease variant according to the first aspect, the method comprising:
a) cultivating the recombinant host cell of the third aspect under conditions suitable for expression of the variant; and
b) recovering the variant.

In a sixth aspect, the present invention relates to a method of degrading a polyester containing material, the method comprising:
a) contacting the polyester containing material with a variant of the first aspect; and, optionally
b) recovering the resulting monomers and/or oligomers.

In a seventh aspect, the present invention relates to a plastic material or product comprising (a) a protease variant according to the first aspect; and (b) at least one polyester; preferably PLA.

In an eighth aspect, the present invention relates to a masterbatch composition comprising (a) a protease variant of the first aspect; and (b) at least one polyester, preferably PLA.

In a ninth aspect, the present invention relates to a method for producing a plastic material or product of the seventh aspect or a masterbatch composition of the eighth aspect, the method comprising:
a) providing a variant of the first aspect; and
b) mixing the variant with at least one polyester at a temperature at which the polyester is in a partially or totally molten state, preferably by extrusion, e.g., wherein the at least one polyester comprises or consists of PLA.

In a tenth aspect, the present invention relates to use of a variant according to the first aspect for degradation of a polyester containing material, preferably a PLA containing material.

### BRIEF DESCRIPTION OF THE FIGURES

FIG. 1 shows an alignment between SEQ ID NO:1 and SEQ ID NO:2, based on Table 1 of WO 1989/06279, from which position numbers corresponding to positions of SEQ ID NO:2 may be readily determined.

FIG. 2 shows the change of turbidity over time, ΔT(t), for a subset of protease variants evaluated in the PLA turbidity assay. All protease variants were dosed at same level of 50 μg enzyme protein, allowing ΔT(t) to be compared and improvement factors to be calculated.

### DEFINITIONS

Protease: The term “protease” means an enzyme that hydrolyses peptide bonds. It includes any enzyme belonging to the EC 3.4 enzyme group (including each of the thirteen subclasses thereof (http://en.wikipedia.org/wiki/Category:EC_3.4). The EC number refers to Enzyme Nomenclature 1992 from NC-IUBMB, Academic Press, San Diego, California, including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The term “subtilases” refer to a sub-group of serine protease according to Siezen et al., Protein Eng. 4 (1991) 719-737 and Siezen et al. Protein Science 6 (1997) 501-523. Serine proteases or serine peptidases is a subgroup of proteases characterized by having a serine in the active site, which forms a covalent adduct with the substrate. Further, the subtilases (and the serine proteases) are characterized by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Protease variants of the invention are endopeptidases (EC 3.4.21). For purposes of the present invention, protease activity is determined according to the procedure described in the Examples below. The variants of the invention exhibit both protease activity and polyester degrading activity. In one aspect, the variants of the present invention have at least 10%, e.g., at least 20%, at least 30%, at least 40%, at least 50%, at least 60%, at least 70%, at least 80%, at least 90%, at least 95%, or at least 100% of the protease activity. In one aspect, the variants of the present invention have at least 10%, e.g., at least 20%, at least 30%, at least 40%, at least 50%, at least 60%, at least 70%, at least 80%, at least 90%, at least 95%, or at least 100% of the protease activity. In one aspect, the variants of the present invention have at least 10%, e.g., at least 20%, at least 30%, at least 40%, at least 50%, at least 60%, at least 70%, at least 80%, at least 90%, at least 95%, or at least 100% of the protease activity of the polypeptide of SEQ ID NO:4.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has polyester degrading activity, preferably PLA degrading activity, and optionally protease activity.

Fusion polypeptide: The term “fusion polypeptide” is a polypeptide in which one polypeptide is fused at the N-terminus or the C-terminus of a variant of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, polyester degrading activity, polyester specificity, proteolytic stability, solubility, specific activity, stability under storage conditions, substrate binding, substrate cleavage, substrate specificity, substrate stability, surface properties, thermal activity, and thermostability.

In one aspect, the variants of the invention have improved polyester degrading activity, in particular improved PLA degrading activity. Polyester degrading activity may be evaluated using the turbidity assay described below.

In one aspect, the variants of the invention have improved thermostability. Thermostability may be evaluated by differential scanning calorimetry for determination of the thermal denaturation temperature, Tm, as described below.

In one aspect, the variants of the invention have improved proteolytic stability. The proteolytic stability conferred by a given substitution may be evaluated by exposing a variant comprising said substitution to proteolytic degradation (e.g., by incubation the variant with a protease or during expression of the variant in a recombinant host cell) and comparing the resulting degradation fragments to the degradation fragments obtained by proteolytic degradation of a variant not comprising said substitution via SDS-PAGE using methods well-known in the art. In one aspect, the variants of the invention have improved polyester specificity, in particular PLA specificity. Specificity may be assessed by determining the ratio between polyester activity and protease activity. Polyester activity and protease activity may be determined according to the procedures describes below.

In one aspect, the variants of the invention have improved solubility.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Mature polypeptide: The term “mature polypeptide” means a polypeptide in its mature form following N-terminal processing (e.g., removal of signal peptide).

Mature polypeptide coding sequence: The term “mature polypeptide coding sequence” means a polynucleotide that encodes a mature polypeptide having polyester degrading activity.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent protease: The term “parent” or “parent protease” means a protease to which an alteration is made to produce the enzyme variants of the present invention. The parent may be a naturally occurring (wild-type) polypeptide or a variant or fragment thereof.

Polyester: The term “polyester” encompasses a group of polymers comprising polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly(ethylene adipate) (PEA) and blends/mixtures of these polymers.

Polyester containing material: The term “polyester containing material” means a material, such as a plastic material, comprising at least one polyester in crystalline, semicrystalline or totally amorphous form. In a particular embodiment, the polyester containing material refers to any item made from at least one plastic material, such as plastic sheet, tube, rod, profile, shape, film, massive block, fiber, textile, etc., which contains at least one polyester, and possibly other substances or additives, such as plasticizers, mineral or organic fillers. In another particular embodiment, the polyester containing material refers to textile or fabrics comprising at least one polyester containing fiber. In another particular embodiment, the polyester containing material refers to a plastic compound, or plastic formulation, in a molten or solid state, suitable for making a plastic product.

Polymer: The term “polymer” means a chemical compound or mixture of compounds whose structure is constituted of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers). According to the invention, the term “oligomers”, when used in reference to a polymer, means molecules containing from 2 to about monomers.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the −nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Subsequence: The term “subsequence” means a polynucleotide having one or more nucleotides absent from the 5′ and/or 3′ end of a mature polypeptide coding sequence; wherein the subsequence encodes a fragment having polyester degrading activity, preferably PLA degrading activity, and optionally protease activity.

Variant and protease variant: The terms “variant” and “protease variant” means a polypeptide having polyester degrading activity, preferably PLA degrading activity, comprising a substitution, an insertion, and/or a deletion, at one or more (e.g., several) positions compared to the parent. A substitution means replacement of the amino acid occupying a position with a different amino acid; a deletion means removal of the amino acid occupying a position; and an insertion means adding an amino acid adjacent to and immediately following the amino acid occupying a position. For purposes of the present invention, polyester degrading activity, in particular PLA degrading activity, is determined according to the procedure described in the Examples below.

Wild-type: The term “wild-type” in reference to an amino acid sequence or nucleic acid sequence means that the amino acid sequence or nucleic acid sequence is a native or naturally occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Protease Variants

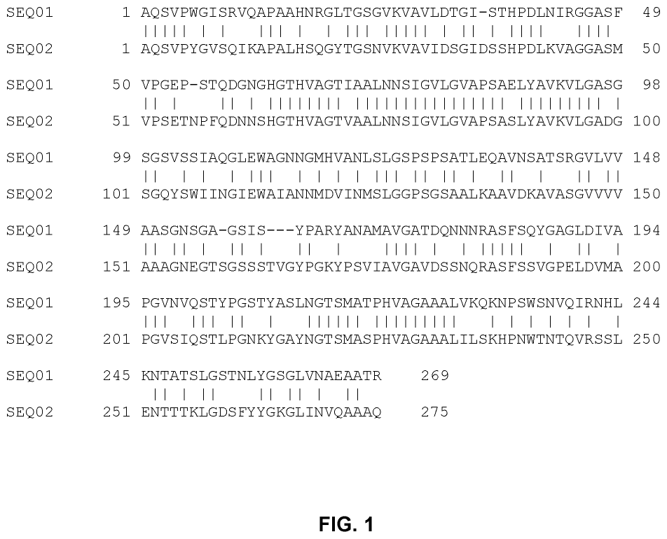

For purposes of the present invention, the polypeptide of SEQ ID NO:2 is used to determine the corresponding amino acid residue number in SEQ ID NO:1 and variants thereof. The amino acid sequence of a SEQ ID NO:1 or variants thereof is aligned with SEQ ID NO:2, and based on the alignment, the amino acid position number corresponding to any amino acid residue in a variant of SEQ ID NO:1 may be determined. Specifically for SEQ ID NO:1 and variants thereof, the numbering is based on the alignment in Table 1 of WO 1989/06279, which shows an alignment of five proteases, including the mature polypeptide of the subtilase BPN′ (BASBPN) sequence (sequence c in the table) and the mature polypeptide of subtilisin 309 from Bacillus clausii, also known as Savinase® (BLSAVI) (sequence a in the table). The accompanying FIG. 1 is provided for reference purposes and shows an alignment between SEQ ID NO:1 and SEQ ID NO:2, based on Table 1 of WO 1989/06279, from which position numbers corresponding to positions of SEQ ID NO:2 may be readily determined. Persons skilled in the art will know that position numbers used for subtilisin 309 and other proteases in the patent literature are often based on the corresponding position numbers of BPN′.

For any other protease (e.g., SEQ ID NO:3 and variants thereof and SEQ ID NO:4 and variants thereof), amino acid position numbers corresponding to any amino acid residue in SEQ ID NO:1 or variants thereof may be determined by alignment to SEQ ID NO:2 using the Needleman-Wunsch algorithm as described above. For clarity, the table below provides an overview of amino acid residues that are substituted in variants of SEQ ID NO:1 (using BPN′ numbering based on the alignment in Table 1 of WO 1989/06279) and the corresponding amino acid residues in variants of SEQ ID NO:3 and variants of SEQ ID NO:4 (using BPN′ numbering based on the Needleman-Wunsch algorithm), with actual substitutions given in parenthesis, and multiple substitutions at the same position being separated by “/”, e.g., S161W/R/Y/L/V.

| SEQ ID NO: 1 | SEQ ID NO: 3 | SEQ ID NO: 4 |
|---|---|---|
| (BPN′ numbering, WO | (BPN′ numbering, | (BPN′ numbering, |
| 1989/06279) | Needleman-Wunsch) | Needleman-Wunsch) |
| S3 (S3T) | T3 (N/A) | T3 (N/A) |
| S9 (S9E) | P9 (P9E) | T9 (T9E) |
| N43 (N43R) | N43 (N43R) | T43 (T43R) |
| N76 (N76D) | D76 (N/A) | N76 (N76D) |
| G97 (G97D) | N97 (N97D) | G97 (G97D) |
| S99 (S99F) | S99 (S99F) | N99 (N98F) |
| S101 (S101L) | S101 (S101L) | R101 (R101L) |
| S103 (S103T) | S103 (S103T) | S103 (S102T) |
| V104 (V104I) | Y104 (Y104I) | V104 (V104I) |
| G127 (G127I) | G127 (G127I) | G127 (G127I) |
| S161 (S161W/R/Y/L/V) | N161 (N161W/Y/L/V) | N/A |
| S163 (S163R) | N163 (N163R) | G163 (G163R) |
| A172 (A172K) | D172 (D171K) | A172 (A172K) |
| A174 (A174G/V) | V174 (V174G) | A174 (A174G/V) |
| M175 (M175A/Y/D/T/V/I) | I175 (I175A/Y/D/T/V) | M175 (M175A/Y/D/T/V/I |
| A176 (A176S) | A176 (A176S) | A176 (A176S) |
| A194 (A194P) | A194 (A194P) | T194 (T194P) |
| V205 (V205I) | V205 (V205I) | I205 (N/A) |
| Q206 (Q206L) | Y206 (Y206L) | Q206 (Q206L) |
| Y209 (Y209W) | Y209 (Y209W) | Y209 (Y209W) |
| A215 (A215K) | A215 (A215K) | A215 (A215K) |
| S259 (S259D) | P259 (P259D) | N259 (N259D) |
| N261 (N261W) | F261 (F261W) | S261 (S261W) |
| L262 (L262E) | Y262 (Y262E) | Q262 (Q262E) |

In describing the variants of the present invention, the nomenclature described below is adapted for ease of reference. The accepted IUPAC single-letter or three-letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple substitutions are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively. Alternatively, multiple substitutions may be separated by commas (“,”), e.g., “Gly205Arg,Ser411Phe” or “G205R,S411F”.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”. Alternatively, multiple deletions may be separated by commas (“,”), e.g., “Gly195*,Ser411*” or “G195*,S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by commas (“,”), e.g., “Arg170Tyr,Gly195Glu” or “R170Y,G195E”.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### SEQUENCE OVERVIEW

SEQ ID NO:1 is the amino acid sequence of the Savinase® protease.
SEQ ID NO:2 is the amino acid sequence of the BPN′ protease.
SEQ ID NO:3 is the amino acid sequence of the Preferenz® P300 protease.
SEQ ID NO:4 is the amino acid sequence of a protease from Bacillus gibsonii.
SEQ ID NO:5 is the amino acid sequence of a variant of SEQ ID NO:1 having improved thermostability compared to SEQ ID NO:1.
SEQ ID NOs:6-66 are the amino acid sequences of variants of SEQ ID NO:1 having improved polyester degrading activity.
SEQ ID NOs:67-86 are the amino acid sequences of variants of SEQ ID NO:3 having improved polyester degrading activity.
SEQ ID NOs:87-91 are the amino acid sequences of variants of SEQ ID NO:4 having improved polyester degrading activity.

### DETAILED DESCRIPTION OF THE INVENTION

The present invention provides new protease variants that exhibit improved polyester degrading activity compared to the parent protease. The protease variants of the invention are particularly useful in processes for degrading plastic materials and plastic products containing polyester(s), such as plastic materials and products containing polylactic acid (PLA). Therefore, the present invention further provides processes for degrading plastic materials and products containing polyester(s), preferably polylactic acid (PLA), using a protease variant of the invention.

Protease Variants with Polyester Degrading Activity

The present invention relates to protease variants having a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to SEQ ID NO:1; wherein the variant comprises the substitutions X99F and X101L; wherein the variant further comprises at least one, e.g., at least two, at least three, at least four, at least five, at least six, at least seven, at least eight, at least nine, or at least ten, or more, substitution selected from the group consisting of X3T, X97D, X103T, X104I, X127I, X161W, X161R, X161Y, X161L, X161V, X163R, X172K, X174G, X174V, X175A, X175Y, X175D, X175T, X175V, X175I, X176S, X194P, and X215K; wherein the variant has polyester degrading activity and, optionally, protease activity; and wherein position numbers are based on the numbering of SEQ ID NO:2.

The variants comprise the substitutions X99F and X101L. These substitutions confer improved polyester degrading activity, in particular PLA degrading activity, to the variants.

In one embodiment, the variants comprise the substitution X3T. This substitution confers improved thermostability to the variants.

In one embodiment, the variants comprise the substitution(s) X103T and/or X104I. These substitutions confer improved polyester degrading activity, in particular PLA degrading activity, to the variants.

In one embodiment, the variants comprise the substitution X127I. This substitution confers improved polyester degrading activity, in particular PLA specificity, to the variants.

In one embodiment, the variant comprises a substitution at position X161, preferably a substitution selected from the group consisting of X161W, X161R, X161Y, X161L, and X161V. These substitutions confer improved polyester degrading activity, in particular PLA degrading activity, to the variants.

In one embodiment, the variants comprise the substitution X163R. This substitution confers improved polyester degrading activity, in particular PLA degrading activity, to the variants.

In one aspect, the variants comprise the substitution X172K. This substitution confers improved polyester degrading activity, in particular PLA degrading activity, to the variants.

In one embodiment, the variant comprises a substitution at position X175, preferably a substitution selected from the group consisting of X175A, X175Y, X175D, X175T, X175V, and X175I. These substitutions confer improved proteolytic stability to the variants.

In one embodiment, the variants comprise the substitution X194P. This substitution confers improved thermostability to the variants.

In one embodiment, the variants comprise the substitution X215K. This substitution confers improved solubility and improved polyester degrading activity, in particular PLA degrading activity, to the variants.

In a preferred embodiment, the variant comprises at least one, e.g., at least two, at least three, at least four, or five, substitution(s) selected from the group consisting of X103T, X104I, X127I, X194P, and X215K.

In one embodiment, the variant comprises the substitutions:
a) X99F, X101L, and X103T;
b) X99F, X101L, and X104I;
c) X99F, X101L, and X127I;
d) X99F, X101L, and X194P;
e) X99F, X101L, and X215K;
f) X99F, X101L, X103T, and X104I;
g) X99F, X101L, X103T, X104I, and X194P;
h) X99F, X101L, X103T, X104I, and X215K;
i) X99F, X101L, X103T, X104I, X194P, and X215K; or
j) X99F, X101L, X103T, X104I, X127I, X194P, and X215K.

In one embodiment, the variants further comprises at least three, e.g., at least four, at least five, at least six, at least seven, at least eight, or nine, substitutions selected from the group consisting of X9E, X43R, X76D, X205I, X206L, X209W, X259D, X261W, and X262E. These substitutions confer, individually and in various combinations, increased thermostability to the variants to SEQ ID NO:1. Preferably, the variants comprise the substitutions X9E, X43R, X76D, X205I, X206L, X209W, X259D, X261W, and X262E.

In a preferred embodiment, the variant comprises the substitutions:
a) X9E, X43R, X76D, X99F, X101L, X205I, X206L, X209W, X215L, X259D, X261W, and X262E;
b) X9E, X43R, X76D, X99F, X101L, X127I, X205I, X206L, X209W, X259D, X261W, and X262E;
c) X9E, X43R, X76D, X99F, X101L, X127I, X205I, X206L, X209W, X215K, X259D, X261W, and X262E;
d) X9E, X43R, X76D, X99F, X101L, X103T, X127I, X205I, X206L, X209W, X215K, X259D, X261W, and X262E;
e) X9E, X43R, X76D, X99F, X101L, X104I, X127I, X205I, X206L, X209W, X215K, X259D, X261W, and X262E;
f) X9E, X43R, X76D, X99F, X101L, X103T, X104I, X205I, X206L, X209W, X215K, X259D, X261W, and X262E
g) X9E, X43R, X76D, X99F, X101L, X103T, X104I, X127I, X205I, X206L, X209W, X215K, X259D, X261W, and X262E; or
h) X9E, X43R, X76D, X99F, X101L, X103T, X104I, X127I, X194P, X205I, X206L, X209W, X215K, X259D, X261W, and X262E.

In one embodiment, the variant further comprises a substitution selected from the group consisting of X161W, X161R, X161Y, X161L, and X161V, and/or a substitution selected from the group consisting of X175A, X175Y, X175D, X175T, X175V, and X175I.

In one embodiment, the variant further comprises at least one, e.g., at least two, at least three, or four, substitution(s) selected from the group consisting of X97D, X172K, X174G, X174V, and X176S.

In one aspect, the present invention relates to a variant of SEQ ID NO:1, wherein the variant has a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to SEQ ID NO:1; wherein the variant comprises the substitutions S99F and S101L; wherein the variant further comprises at least one, e.g., at least two, at least three, at least four, at least five, at least six, at least seven, at least eight, at least nine, or at least ten, at least eleven, at least twelve, or thirteen, substitution(s) selected from the group consisting of S3T, G97D, S103T, V104I, G127I, S161W, S161R, S161Y, S161L, S161V, S163R, A172K, A174G, A174V, M175A, M175Y, M175D, M175T, M175V, M175I, A176S, A194P, and A215K; wherein the variant has polyester degrading activity and, optionally, protease activity; and wherein position numbers are based on the numbering of SEQ ID NO:2.

In a preferred embodiment, the variant comprises at least one, e.g., at least two, at least three, at least four, or five substitution(s) selected from the group consisting of S103T, V104I, G127I, A194P, and A215K.

In one embodiment, the variant comprises SEQ ID NO:1 with the substitutions:
a) S99F, S101L, and S103T;
b) S99F, S101L, and V104I;
c) S99F, S101 L, and G127I;
d) S99F, S101L, and A194P;
e) S99F, S101L, and A215K;
f) S99F, S101L, S103T, and V104I;
g) S99F, S101L, S103T, V104I, and A194P;
h) S99F, S101L, S103T, V104I, and A215K;
i) S99F, S101L, S103T, V104I, A194P, and A215K; or
j) S99F, S101L, S103T, V104I, G127I, A194P, and A215K.

In one embodiment, the variant further comprises at least three, e.g., at least four, at least five, at least six, at least seven, at least eight, or nine, substitutions selected from the group consisting of S9E, N43R, N76D, V205I, Q206L, Y209W, S259D, N261W, and L262E. These substitutions confer, individually and in various combinations, increased thermostability compared to SEQ ID NO:1. Preferably, the variant further comprises the substitutions S9E, N43R, N76D, V205I, Q206L, Y209W, S259D, N261W, and L262E.

In one embodiment, the variant comprises SEQ ID NO:1 with the substitutions S9E, N43R, N76D, S99F, S101L, V205I, Q206L, Y209W, S259D, N261W, and L262E and at least one, e.g., at least two, at least three, at least four, at least five, at least six, at least seven, at least eight, at least nine, at least ten, at least eleven, at least twelve, or thirteen, substitutions, selected from the group consisting of S3T, G97D, S103T, V104I, G127I, S161W, S161R, S161Y, S161L, S161V, S163R, A172K, A174G, A174V, M175A, M175Y, M175D, M175T, M175V, M175I, A176S, A194P, and A215K.

In a preferred embodiment, the variant comprises SEQ ID NO:1 with the substitutions:
a) S9E, N43R, N76D, S99F, S101L, V205I, Q206L, Y209W, A215K, S259D, N261W, and L262E;
b) S9E, N43R, N76D, S99F, S101L, G127I, V205I, Q206L, Y209W, S259D, N261W, and L262E;
c) S9E, N43R, N76D, S99F, S101L, G127I, V205I, Q206L, Y209W, A215K, S259D, N261W, and L262E;
d) S9E, N43R, N76D, S99F, S101L, S103T, G127I, V205I, Q206L, Y209W, A215K, S259D, N261W, and L262E;
e) S9E, N43R, N76D, S99F, S101L, V104I, G127I, V205I, Q206L, Y209W, A215K, S259D, N261W, and L262E;
f) S9E, N43R, N76D, S99F, S101L, S103T, V104I, V205I, Q206L, Y209W, A215K, S259D, N261W, and L262E
g) S9E, N43R, N76D, S99F, S101L, S103T, V104I, G127I, V205I, Q206L, Y209W, A215K, S259D, N261W, and L262E; or
h) S9E, N43R, N76D, S99F, S101L, S103T, V104I, G127I, A194P, V205I, Q206L, Y209W, A215K, S259D, N261W, and L262E.

In one embodiment, the variant further comprises a substitution selected from the group consisting of S161W, S161R, S161Y, S161L, and S161V, and/or a substitution selected from the group consisting of M175A, M175Y, M175D, M175T, M175V, and M175I.

In one embodiment, the variant further comprises at least one, e.g., at least two, at least three, or four, substitution(s) selected from the group consisting of G97D, A172K, A174G, A174V, and A176S.

In one aspect, the present invention relates to a variant of SEQ ID NO:3, wherein the variant has a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to SEQ ID NO:3; wherein the variant comprises the substitutions S99F and S101L; wherein the variant further comprises at least one, e.g., at least two, at least three, at least four, at least five, at least six, at least seven, at least eight, at least nine, or at least ten, at least eleven, or twelve, substitution(s) selected from the group consisting of N97D, S103T, Y104I, G127I, N161W, N161Y, N161L, N161V, N163R, D172K, V174G, I175A, I175Y, I175D, I175T, I175V, A176S, A194P, and A215K; wherein the variant has polyester degrading activity and, optionally, protease activity; and wherein position numbers are based on the numbering of SEQ ID NO:2.

In a preferred embodiment, the variant comprises at least one, e.g., at least two, at least three, at least four, or five substitution(s) selected from the group consisting of S103T, Y104I, G127I, A194P, and A215K.

In one embodiment, the variant comprises SEQ ID NO:3 with the substitutions:
a) S99F, S101L, and S103T;
b) S99F, S101L, and Y104I;
c) S99F, S101 L, and G127I;
d) S99F, S101L, and A194P;
e) S99F, S101L, and A215K;
f) S99F, S101L, S103T, and Y104I;
g) S99F, S101L, S103T, Y104I, and A194P;
h) S99F, S101L, S103T, Y104I, and A215K;
i) S99F, S101L, S103T, Y104I, A194P, and A215K; or
j) S99F, S101L, S103T, Y104I, G127I, A194P, and A215K.

In one embodiment, the variant further comprises at least three, e.g., at least four, at least five, at least six, at least seven, or eight, substitutions selected from the group consisting of P9E, N43R, V205I, Y206L, Y209W, P259D, F261W, and Y262E. These substitutions confer, individually and in various combinations, increased thermostability to the variants to SEQ ID NO:3. Preferably, the variants further comprise the substitutions P9E, N43R, V205I, Y206L, Y209W, P259D, F261W, and Y262E.

In one embodiment, the variant further comprises at least three, e.g., at least four, at least five, at least six, at least seven, or eight, substitutions selected from the group consisting of P9E, N43R, V205I, Y206L, Y209W, P259D, F261W, and Y262E. These substitutions confer, individually and in various combinations, increased thermostability compared to SEQ ID NO:3. Preferably, the variant further comprises the substitutions P9E, N43R, V205I, Y206L, Y209W, P259D, F261W, and Y262E.

In one embodiment, the variant comprises SEQ ID NO:3 with the substitutions P9E, N43R, V205I, Y206L, Y209W, P259D, F261W, and Y262E and at least one, e.g., at least two, at least three, at least four, at least five, at least six, at least seven, at least eight, at least nine, at least ten, at least eleven, or twelve, substitution(s), selected from the group consisting of N97D, S103T, Y104I, G127I, N161W, N161Y, N161L, N161V, N163R, D172K, V174G, I175A, I175Y, I175D, I175T, I175V, A176S, A194P, and A215K.

In a preferred embodiment, the variant comprises SEQ ID NO:3 with the substitutions:
a) P9E, N43R, S99F, S101L, V205I, Y206L, Y209W, A215K, P259D, F261W, and Y262E;
b) P9E, N43R, S99F, S101L, G127I, V205I, Y206L, Y209W, P259D, F261W, and Y262E;
c) P9E, N43R, S99F, S101L, G127I, V205I, Y206L, Y209W, A215K, P259D, F261W, and Y262E;
d) P9E, N43R, S99F, S101L, S103T, G127I, V205I, Y206L, Y209W, A215K, P259D, F261W, and Y262E;
e) P9E, N43R, S99F, S101L, V104I, G127I, V205I, Y206L, Y209W, A215K, P259D, F261W, and Y262E;
f) P9E, N43R, S99F, S101L, S103T, V104I, V205I, Y206L, Y209W, A215K, P259D, F261W, and Y262E
g) P9E, N43R, S99F, S101L, S103T, V104I, G127I, V205I, Y206L, Y209W, A215K, P259D, F261W, and Y262E; or
h) P9E, N43R, S99F, S101L, S103T, V104I, G127I, A194P, V205I, Y206L, Y209W, A215K, P259D, F261W, and Y262E.

In one embodiment, the variant further comprises a substitution selected from the group consisting of N161W, N161R, N161Y, N161L, and N161V, and/or a substitution selected from the group consisting of I175A, I175Y, I175D, I175T, and I175V.

In one embodiment, the variant further comprises at least one, e.g., at least two, at least three, or four, substitution(s) selected from the group consisting of N97D, D172K, V174G, and A176S.

In one aspect, the present invention relates to a variant of SEQ ID NO:4, wherein the variant has a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to SEQ ID NO:4; wherein the variant comprises the substitutions N99F and R101L; wherein the variant further comprises at least one, e.g., at least two, at least three, at least four, at least five, at least six, at least seven, at least eight, at least nine, or at least ten, or eleven, substitution(s) selected from the group consisting of G97D, S103T, V104I, G127I, G163R, A172K, A174G, A174V, M175A, M175Y, M175D, M175T, M175V, M175I, A176S, T194P, and A215K; wherein the variant has polyester degrading activity and, optionally, protease activity; and wherein position numbers are based on the numbering of SEQ ID NO:4.

In a preferred embodiment, the variant comprises at least one, e.g., at least two, at least three, at least four, or five substitution(s) selected from the group consisting of S103T, V104I, G127I, T194P, and A215K.

In one embodiment, the variant comprises SEQ ID NO:4 with the substitutions:
a) N99F, R101L, and S103T;
b) N99F, R101L, and V104I;
c) N99F, R101L, and G127I;
d) N99F, R101L, and T194P;
e) N99F, R101L, and A215K;
f) N99F, R101L, S103T, and V104I;
g) N99F, R101L, S103T, V104I, and T194P;
h) N99F, R101L, S103T, V104I, and A215K;
i) N99F, R101L, S103T, V104I, T194P, and A215K; or
j) N99F, R101L, S103T, V104I, G127I, T194P, and A215K.

In one embodiment, the variant further comprises at least three, e.g., at least four, at least five, at least six, at least seven, or eight, substitutions selected from the group consisting of T9E, T43R, N76D, Q206L, Y209W, N259D, S261W, and Q262E. These substitutions confer, individually and in various combinations, increased thermostability compared to SEQ ID NO:4. Preferably, the variant further comprises the substitutions T9E, T43R, N76D, Q206L, Y209W, N259D, S261W, and Q262E.

In one embodiment, the variant comprises SEQ ID NO:1 with the substitutions T9E, T43R, N76D, N99F, R101L, Q206L, Y209W, N259D, S261W, and Q262E and at least one, e.g., at least two, at least three, at least four, at least five, at least six, at least seven, at least eight, at least nine, at least ten, or eleven, substitution(s), selected from the group consisting of G97D, S103T, V104I, G127I, G163R, A172K, A174G, A174V, M175A, M175Y, M175D, M175T, M175V, M175I, A176S, T194P, and A215K.

In a preferred embodiment, the variant comprises SEQ ID NO:4 with the substitutions:
a) T9E, T43R, N76D, N99F, R101L, Q206L, Y209W, A215K, N259D, S261W, and Q262E;
b) T9E, T43R, N76D, N99F, R101L, G127I, Q206L, Y209W, N259D, S261W, and Q262E;
c) T9E, T43R, N76D, N99F, R101L, G127I, Q206L, Y209W, A215K, N259D, S261W, and Q262E;
d) T9E, T43R, N76D, N99F, R101L, S103T, G127I, Q206L, Y209W, A215K, N259D, S261W, and Q262E;
e) T9E, T43R, N76D, N99F, R101L, V104I, G127I, Q206L, Y209W, A215K, N259D, S261W, and Q262E;
f) T9E, T43R, N76D, N99F, R101L, S103T, V104I, Q206L, Y209W, A215K, N259D, S261W, and Q262E
g) T9E, T43R, N76D, N99F, R101L, S103T, V104I, G127I, Q206L, Y209W, A215K, N259D, S261W, and Q262E; or
h) T9E, T43R, N76D, N99F, R101L, S103T, V104I, G127I, T194P, Q206L, Y209W, A215K, N259D, S261W, and Q262E.

In one embodiment, the variant further comprises a substitution selected from the group consisting of M175A, M175Y, M175D, M175T, M175V, and M175I.

In one embodiment, the variant further comprises at least one, e.g., at least two, at least three, or four, substitution(s) selected from the group consisting of G97D, A172K, A174G, A174V, and A176S.

In one aspect, the variant of the inventions comprises or consists of SEQ ID NO:1 with the substitutions selected from the group consisting of:
a) S9E, N43R, N76D, S99F, S101L, V205I, Q206L, Y209W, S259D, N261W, and L262E;
b) S9E, N43R, N76D, S99F, S101L, G127I, V205I, Q206L, Y209W, S259D, N261W, and L262E;
c) S9E, N43R, N76D, S99F, S101L, G127I, V205I, Q206L, Y209W, A215K, S259D, N261W, and L262E;
d) S9E, N43R, N76D, S99F, S101L, S103T, G127I, V205I, Q206L, Y209W, A215K, S259D, N261W, and L262E;
e) S9E, N43R, N76D, S99F, S101L, V104I, G127I, V205I, Q206L, Y209W, A215K, S259D, N261W, and L262E;
f) S9E, N43R, N76D, S99F, S101L, S103T, V104I, G127I, V205I, Q206L, Y209W, A215K, S259D, N261W, and L262E;
g) S9E, N43R, N76D, S99F, S101L, S103T, V104I, G127I, A194P, V205I, Q206L, Y209W, A215K, S259D, N261W, and L262E;
h) S3T, S9E, N43R, N76D, S99F, S101L, S103T, V104I, G127I, A194P, V205I, Q206L, Y209W, A215K, S259D, N261W, and L262E.
i) S3T, S9E, N43R, N76D, S99F, S101L, S103T, V104I, G127I, A194P, V205I, Q206L, Y209W, A215K, S259D, N261W, L262E, and a substitution selected from the group consisting of M175A, M175Y, M175D, M175T, M175V, and M175I; and
l) S3T, S9E, N43R, N76D, S99F, S101L, S103T, V104I, G127I, A194P, V205I, Q206L, Y209W, A215K, S259D, N261W, L262E, and a substitution selected from the group consisting of S161W, S161R, S161Y, S161L, and S161.

In one aspect, the variant is selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, and SEQ ID NO:91.

In one aspect, the variant comprises or consists of SEQ ID NO:1 with the substitutions S9E, N43R, N76D, S99F, S101L, S103T, V104I, V205I, Q206L, Y209W, S259D, N261W, L262E. Preferably, the variant comprises or consists of SEQ ID NO:6.

In one aspect, the variant comprises or consists of SEQ ID NO:1 with the substitutions S9E, N43R, N76D, S99F, S101L, S103T, V104I, S163R, V205I, Q206L, Y209W, S259D, N261W, L262E. Preferably, the variant comprises or consists of SEQ ID NO:7.

In one aspect, the variant comprises or consists of SEQ ID NO:1 with the substitutions S9E, N43R, N76D, S99F, S101L, S103T, V104I, S156E, S163R, V205I, Q206L, Y209W, S259D, N261W, L262E. Preferably, the variant comprises or consists of SEQ ID NO:8.

In one aspect, the variant comprises or consists of SEQ ID NO:1 with the substitutions S9E, N43R, N76D, S99F, S101L, V104I, S156E, S163R, V205I, Q206L, Y209W, S259D, N261W, L262E. Preferably, the variant comprises or consists of SEQ ID NO:9.

In one aspect, the variant comprises or consists of SEQ ID NO:1 with the substitutions S9E, N43R, N76D, S101L, V104I, V205I, Q206L, Y209W, S259D, N261W, L262E. Preferably, the variant comprises or consists of SEQ ID NO:10.

In one aspect, the variant comprises or consists of SEQ ID NO:1 with the substitutions S9E, N43R, N76D, S99F, S101L, S156E, S163R, V205I, Q206L, Y209W, S259D, N261W, L262E. Preferably, the variant comprises or consists of SEQ ID NO:11.

In one aspect, the variant comprises or consists of SEQ ID NO:1 with the substitutions S9E, N43R, N76D, S99F, S101L, S103T, V104I, V205I, Q206L, Y209W, A215K, S259D, N261W, L262E. Preferably, the variant comprises or consists of SEQ ID NO:12.

In one aspect, the variant comprises or consists of SEQ ID NO:1 with the substitutions S3T, S9E, N43R, N76D, S99F, S101L, S103T, V104I, A194P, V205I, Q206L, Y209W, A215K, S259D, N261W, L262E. Preferably, the variant comprises or consists of SEQ ID NO:13.

In one aspect, the variant comprises or consists of SEQ ID NO:1 with the substitutions S9E, N43R, N76D, S99F, S101L, S103T, V104I, S163R, V205I, Q206L, Y209W, A215K, S259D, N261W, L262E. Preferably, the variant comprises or consists of SEQ ID NO:14.

In one aspect, the variant comprises or consists of SEQ ID NO:1 with the substitutions S9E, N43R, N76D, S99F, S101L, S103T, V104I, G127I, V205I, Q206L, Y209W, A215K, S259D, N261W, L262E. Preferably, the variant comprises or consists of SEQ ID NO:15.

In one aspect, the variant comprises or consists of SEQ ID NO:1 with the substitutions S3T, S9E, N43R, N76D, S99F, S101L, S103T, V104I, G127I, V205I, Q206L, Y209W, A215K, S259D, N261W, L262E. Preferably, the variant comprises or consists of SEQ ID NO:16.

In one aspect, the variant comprises or consists of SEQ ID NO:1 with the substitutions S9E, N43R, N76D, S99F, S101L, S103T, V104I, G127I, A194P, V205I, Q206L, Y209W, A215K, S259D, N261W, L262E. Preferably, variant comprises or consists of SEQ ID NO:17.

In one aspect, the variant comprises or consists of SEQ ID NO:1 with the substitutions S3T, S9E, N43R, N76D, S99F, S101L, S103T, V104I, G127I, A194P, V205I, Q206L, Y209W, A215K, S259D, N261W, L262E. Preferably, the variant comprises or consists of SEQ ID NO:18.

In one aspect, the variant comprises or consists of SEQ ID NO:1 with the substitutions S3T, S9E, N43R, N76D, S99F, S101L, S103T, V104I, A174G, A176S, A194P, V205I, Q206L, Y209W, A215K, S259D, N261W, L262E. Preferably, the variant comprises or consists of SEQ ID NO:19.

In one aspect, the variant comprises or consists of SEQ ID NO:1 with the substitutions S3T, S9E, N43R, N76D, S99F, S101L, S103T, V104I, A172K, A174G, A194P, V205I, Q206L, Y209W, A215K, S259D, N261W, L262E. Preferably, the variant comprises or consists of SEQ ID NO:20.

In one aspect, the variant comprises or consists of SEQ ID NO:1 with the substitutions S3T, S9E, N43R, N76D, S99F, S101L, S103T, V104I, S161W, M175A, A194P, V205I, Q206L, Y209W, A215K, S259D, N261W, L262E. Preferably, the variant comprises or consists of SEQ ID NO:21.

In one aspect, the variant comprises or consists of SEQ ID NO:1 with the substitutions S3T, S9E, N43R, N76D, S99F, S101L, S103T, V104I, S161R, M175A, A194P, V205I, Q206L, Y209W, A215K, S259D, N261W, L262E. Preferably, the variant comprises or consists of SEQ ID NO:22.

In one aspect, the variant comprises or consists of SEQ ID NO:1 with the substitutions S3T, S9E, N43R, N76D, S99F, S101L, S103T, V104I, M175A, A176S, A194P, V205I, Q206L, Y209W, A215K, S259D, N261W, L262E. Preferably, the variant comprises or consists of SEQ ID NO:23.

In one aspect, the variant comprises or consists of SEQ ID NO:1 with the substitutions S3T, S9E, N43R, N76D, S99F, S101L, S103T, V104I, A172K, M175A, A194P, V205I, Q206L, Y209W, A215K, S259D, N261W, L262E. Preferably, the variant comprises or consists of SEQ ID NO:24.

In one aspect, the variant comprises or consists of SEQ ID NO:1 with the substitutions S3T, S9E, N43R, N76D, S99F, S101L, S103T, V104I, S161Y, M175Y, A194P, V205I, Q206L, Y209W, A215K, S259D, N261W, L262E. Preferably, the variant comprises or consists of SEQ ID NO:25.

In one aspect, the variant comprises or consists of SEQ ID NO:1 with the substitutions S3T, S9E, N43R, N76D, S99F, S101L, S103T, V104I, S161L, M175Y, A194P, V205I, Q206L, Y209W, A215K, S259D, N261W, L262E. Preferably, the variant comprises or consists of SEQ ID NO:26.

In one aspect, the variant comprises or consists of SEQ ID NO:1 with the substitutions S3T, S9E, N43R, N76D, S99F, S101L, S103T, V104I, M175Y, A176S, A194P, V205I, Q206L, Y209W, A215K, S259D, N261W, L262E. Preferably, the variant comprises or consists of SEQ ID NO:27.

In one aspect, the variant comprises or consists of SEQ ID NO:1 with the substitutions S3T, S9E, N43R, N76D, S99F, S101L, S103T, V104I, A174V, M175Y, A194P, V205I, Q206L, Y209W, A215K, S259D, N261W, L262E. Preferably, the variant comprises or consists of SEQ ID NO: 28.

In one aspect, the variant comprises or consists of SEQ ID NO:1 with the substitutions S3T, S9E, N43R, N76D, S99F, S101L, S103T, V104I, S161R, M175D, A194P, V205I, Q206L, Y209W, A215K, S259D, N261W, L262E. Preferably, the variant comprises or consists of SEQ ID NO:29.

In one aspect, the variant comprises or consists of SEQ ID NO:1 with the substitutions S3T, S9E, N43R, N76D, S99F, S101L, S103T, V104I, A172K, M175D, A194P, V205I, Q206L, Y209W, A215K, S259D, N261W, L262E. Preferably, the variant comprises or consists of SEQ ID NO:30.

In one aspect, the variant comprises or consists of SEQ ID NO:1 with the substitutions S3T, S9E, N43R, N76D, S99F, S101L, S103T, V104I, S161W, M175T, A194P, V205I, Q206L, Y209W, A215K, S259D, N261W, L262E. Preferably, the variant comprises or consists of SEQ ID NO:31.

In one aspect, the variant comprises or consists of SEQ ID NO:1 with the substitutions S3T, S9E, N43R, N76D, S99F, S101L, S103T, V104I, S161V, M175T, A194P, V205I, Q206L, Y209W, A215K, S259D, N261W, L262E. Preferably, the variant comprises or consists of SEQ ID NO:32.

In one aspect, the variant comprises or consists of SEQ ID NO:1 with the substitutions S3T, S9E, N43R, N76D, S99F, S101L, S103T, V104I, S161Y, M175T, A194P, V205I, Q206L, Y209W, A215K, S259D, N261W, L262E. Preferably, the variant comprises or consists of SEQ ID NO:33.

In one aspect, the variant comprises or consists of SEQ ID NO:1 with the substitutions S3T, S9E, N43R, N76D, S99F, S101L, S103T, V104I, S161R, M175T, A194P, V205I, Q206L, Y209W, A215K, S259D, N261W, L262E. Preferably, the variant comprises or consists of SEQ ID NO:34.

In one aspect, the variant comprises or consists of SEQ ID NO:1 with the substitutions S3T, S9E, N43R, N76D, S99F, S101L, S103T, V104I, S161L, M175T, A194P, V205I, Q206L, Y209W, A215K, S259D, N261W, L262E. Preferably, the variant comprises or consists of SEQ ID NO:35.

In one aspect, the variant comprises or consists of SEQ ID NO:1 with the substitutions S3T, S9E, N43R, N76D, S99F, S101L, S103T, V104I, M175T, A176S, A194P, V205I, Q206L, Y209W, A215K, S259D, N261W, L262E. Preferably, the variant comprises or consists of SEQ ID NO:36.

In one aspect, the variant comprises or consists of SEQ ID NO:1 with the substitutions S3T, S9E, N43R, N76D, S99F, S101L, S103T, V104I, A174G, M175T, A194P, V205I, Q206L, Y209W, A215K, S259D, N261W, L262E. Preferably, the variant comprises or consists of SEQ ID NO:37.

In one aspect, the variant comprises or consists of SEQ ID NO:1 with the substitutions S3T, S9E, N43R, N76D, S99F, S101L, S103T, V104I, A172K, M175T, A194P, V205I, Q206L, Y209W, A215K, S259D, N261W, L262E. Preferably, the variant comprises or consists of SEQ ID NO:38.

In one aspect, the variant comprises or consists of SEQ ID NO:1 with the substitutions S3T, S9E, N43R, N76D, S99F, S101L, S103T, V104I, S161W, M175V, A194P, V205I, Q206L, Y209W, A215K, S259D, N261W, L262E. Preferably, the variant comprises or consists of SEQ ID NO:39.

In one aspect, the variant comprises or consists of SEQ ID NO:1 with the substitutions S3T, S9E, N43R, N76D, S99F, S101L, S103T, V104I, S161Y, M175V, A194P, V205I, Q206L, Y209W, A215K, S259D, N261W, L262E. Preferably, the variant comprises or consists of SEQ ID NO:40.

In one aspect, the variant comprises or consists of SEQ ID NO:1 with the substitutions S3T, S9E, N43R, N76D, S99F, S101L, S103T, V104I, S161R, M175V, A194P, V205I, Q206L, Y209W, A215K, S259D, N261W, L262E. Preferably, the variant comprises or consists of SEQ ID NO:41.

In one aspect, the variant comprises or consists of SEQ ID NO:1 with the substitutions S3T, S9E, N43R, N76D, S99F, S101L, S103T, V104I, S161L, M175V, A194P, V205I, Q206L, Y209W, A215K, S259D, N261W, L262E. Preferably, the variant comprises or consists of SEQ ID NO:42.

In one aspect, the variant comprises or consists of SEQ ID NO:1 with the substitutions S3T, S9E, N43R, N76D, S99F, S101L, S103T, V104I, M175V, A176S, A194P, V205I, Q206L, Y209W, A215K, S259D, N261W, L262E. Preferably, the variant comprises or consists of SEQ ID NO:43.

In one aspect, the variant comprises or consists of SEQ ID NO:1 with the substitutions S3T, S9E, N43R, N76D, S99F, S101L, S103T, V104I, A174G, M175V, A194P, V205I, Q206L, Y209W, A215K, S259D, N261W, L262E. Preferably, the variant comprises or consists of SEQ ID NO:44.

In one aspect, the variant comprises or consists of SEQ ID NO:1 with the substitutions S3T, S9E, N43R, N76D, S99F, S101L, S103T, V104I, A172K, M175V, A194P, V205I, Q206L, Y209W, A215K, S259D, N261W, L262E. Preferably, the variant comprises or consists of SEQ ID NO:45.

In one aspect, the variant comprises or consists of SEQ ID NO:1 with the substitutions S3T, S9E, N43R, N76D, S99F, S101L, S103T, V104I, S161W, M175I, A194P, V205I, Q206L, Y209W, A215K, S259D, N261W, L262E. Preferably, the variant comprises or consists of SEQ ID NO:46.

In one aspect, the variant comprises or consists of SEQ ID NO:1 with the substitutions S3T, S9E, N43R, N76D, S99F, S101L, S103T, V104I, S161V, M175I, A194P, V205I, Q206L, Y209W, A215K, S259D, N261W, L262E. Preferably, the variant comprises or consists of SEQ ID NO:47.

In one aspect, the variant comprises or consists of SEQ ID NO:1 with the substitutions S3T, S9E, N43R, N76D, S99F, S101L, S103T, V104I, S161Y, M175I, A194P, V205I, Q206L, Y209W, A215K, S259D, N261W, L262E. Preferably, the variant comprises or consists of SEQ ID NO:48.

In one aspect, the variant comprises or consists of SEQ ID NO:1 with the substitutions S3T, S9E, N43R, N76D, S99F, S101L, S103T, V104I, S161R, M175I, A194P, V205I, Q206L, Y209W, A215K, S259D, N261W, L262E. Preferably, the variant comprises or consists of SEQ ID NO:49.

In one aspect, the variant comprises or consists of SEQ ID NO:1 with the substitutions S3T, S9E, N43R, N76D, S99F, S101L, S103T, V104I, S161L, M175I, A194P, V205I, Q206L, Y209W, A215K, S259D, N261W, L262E. Preferably, the variant comprises or consists of SEQ ID NO:50.

In one aspect, the variant comprises or consists of SEQ ID NO:1 with the substitutions S3T, S9E, N43R, N76D, S99F, S101L, S103T, V104I, A174G, M175I, A194P, V205I, Q206L, Y209W, A215K, S259D, N261W, L262E. Preferably, the variant comprises or consists of SEQ ID NO:51.

In one aspect, the variant comprises or consists of SEQ ID NO:1 with the substitutions S3T, S9E, N43R, N76D, S99F, S101L, S103T, V104I, A172K, M175I, A194P, V205I, Q206L, Y209W, A215K, S259D, N261W, L262E. Preferably, the variant comprises or consists of SEQ ID NO:52.

In one aspect, the variant comprises or consists of SEQ ID NO:1 with the substitutions S3T, S9E, N43R, N76D, S99F, S101L, S103T, V104I, S163R, A172K, M175I, A194P, V205I, Q206L, Y209W, A215K, S259D, N261W, L262E. Preferably, the variant comprises or consists of SEQ ID NO:53.

In one aspect, the variant comprises or consists of SEQ ID NO:1 with the substitutions S3T, S9E, N43R, N76D, S99F, S101L, S103T, V104I, S161W, A194P, V205I, Q206L, Y209W, A215K, S259D, N261W, L262E. Preferably, the variant comprises or consists of SEQ ID NO:54.

In one aspect, the variant comprises or consists of SEQ ID NO:1 with the substitutions S3T, S9E, N43R, N76D, S99F, S101L, S103T, V104I, S161V, A194P, V205I, Q206L, Y209W, A215K, S259D, N261W, L262E. Preferably, the variant comprises or consists of SEQ ID NO:55.

In one aspect, the variant comprises or consists of SEQ ID NO:1 with the substitutions S3T, S9E, N43R, N76D, S99F, S101L, S103T, V104I, S161Y, A194P, V205I, Q206L, Y209W, A215K, S259D, N261W, L262E. Preferably, the variant comprises or consists of SEQ ID NO:56.

In one aspect, the variant comprises or consists of SEQ ID NO:1 with the substitutions S3T, S9E, N43R, N76D, S99F, S101L, S103T, V104I, S161R, V205I, Q206L, Y209W, A194P, A215K, S259D, N261W, L262E. Preferably, the variant comprises or consists of SEQ ID NO:57.

In one aspect, the variant comprises or consists of SEQ ID NO:1 with the substitutions S3T, S9E, N43R, N76D, S99F, S101L, S103T, V104I, M175T, A194P, V205I, Q206L, Y209W, A215K, S259D, N261W, L262E. Preferably, the variant comprises or consists of SEQ ID NO:58.

In one aspect, the variant comprises or consists of SEQ ID NO:1 with the substitutions S3T, S9E, N43R, N76D, S99F, S101L, S103T, V104I, M175V, A194P, V205I, Q206L, Y209W, A215K, S259D, N261W, L262E. Preferably, the variant comprises or consists of SEQ ID NO:59.

In one aspect, the variant comprises or consists of SEQ ID NO:1 with the substitutions S3T, S9E, N43R, N76D, S99F, S101L, S103T, V104I, M175I, A194P, V205I, Q206L, Y209W, A215K, S259D, N261W, L262E. Preferably, the variant comprises or consists of SEQ ID NO:60.

In one aspect, the variant comprises or consists of SEQ ID NO:1 with the substitutions S3T, S9E, N43R, N76D, S99F, S101L, S103T, V104I, A176S, A194P, V205I, Q206L, Y209W, A125K, S259D, N261W, L262E. Preferably, the variant comprises or consists of SEQ ID NO:61.

In one aspect, the variant comprises or consists of SEQ ID NO:1 with the substitutions S3T, S9E, N43R, N76D, S99F, S101L, S103T, V104I, A174G, A194P, V205I, Q206L, Y209W, A215K, S259D, N261W, L262E. Preferably, the variant comprises or consists of SEQ ID NO:62.

In one aspect, the variant comprises or consists of SEQ ID NO:1 with the substitutions S3T, S9E, N43R, N76D, S99F, S101L, S103T, V104I, A172K, A194P, V205I, Q206L, Y209W, A215K, S259D, N261W, L262E. Preferably, the variant comprises or consists of SEQ ID NO:63.

In one aspect, the variant comprises or consists of SEQ ID NO:1 with the substitutions S3T, S9E, N43R, N76D, S99F, S101L, S103T, V104I, M175A, A194P, V205I, Q206L, Y209W, A215K, S259D, N261W, L262E. Preferably, the variant comprises or consists of SEQ ID NO:64.

In one aspect, the variant comprises or consists of SEQ ID NO:1 with the substitutions S3T, S9E, N43R, N76D, S99F, S101L, S103T, V104I, M175Y, A194P, V205I, Q206L, Y209W, A215K, S259D, N261W, L262E. Preferably, the variant comprises or consists of SEQ ID NO:65.

In one aspect, the variant comprises or consists of SEQ ID NO:1 with the substitutions S3T, S9E, N43R, N76D, S99F, S101L, S103T, V104I, A194P, V205I, Q206L, Y209W, A215K, S259D, N261W, L262E. Preferably, the variant comprises or consists of SEQ ID NO:66.

In one aspect, the variant comprises or consists of SEQ ID NO:3 with the substitutions S99F and S101L. Preferably, the variant comprises or consists of SEQ ID NO:67.

In one aspect, the variant comprises or consists of SEQ ID NO:3 with the substitutions S99F, S101L, and A194P. Preferably, the variant comprises or consists of SEQ ID NO:68.

In one aspect, the variant comprises or consists of SEQ ID NO:3 with the substitutions S99F, S101L, and A215K. Preferably, the variant comprises or consists of SEQ ID NO:69.

In one aspect, the variant comprises or consists of SEQ ID NO:3 with the substitutions S99F, S101L, S103T, and Y104I. Preferably, the variant comprises or consists of SEQ ID NO:70.

In one aspect, the variant comprises or consists of SEQ ID NO:3 with the substitutions S99F, S101L, S156E, and N163R. Preferably, the variant comprises or consists of SEQ ID NO:71.

In one aspect, the variant comprises or consists of SEQ ID NO:3 with the substitutions S99F, S101L, S103T, Y104I, and A194P. Preferably, the variant comprises or consists of SEQ ID NO:72.

In one aspect, the variant comprises or consists of SEQ ID NO:3 with the substitutions S99F, S101L, S156E, N163R, and A194P. Preferably, the variant comprises or consists of SEQ ID NO:73.

In one aspect, the variant comprises or consists of SEQ ID NO:3 with the substitutions S99F, S101L, A194P, and A215K. Preferably, the variant comprises or consists of SEQ ID NO:74.

In one aspect, the variant comprises or consists of SEQ ID NO:3 with the substitutions S99F, S101L, S156E, N163R, and A215K. Preferably, the variant comprises or consists of SEQ ID NO:75.

In one aspect, the variant comprises or consists of SEQ ID NO:3 with the substitutions S99F, S101L, S103T, Y104I, S156E, N163R, and A194P. Preferably, the variant comprises or consists of SEQ ID NO:76.

In one aspect, the variant comprises or consists of SEQ ID NO:3 with the substitutions S99F, S101L, S103T, Y104I, A194P, and A215K. Preferably, the variant comprises or consists of SEQ ID NO:77.

In one aspect, the variant comprises or consists of SEQ ID NO:3 with the substitutions S99F, S101L, S103T, Y104I, S156E, N163R, and A215K. Preferably, the variant comprises or consists of SEQ ID NO:78.

In one aspect, the variant comprises or consists of SEQ ID NO:3 with the substitutions S99F, S101L, S103T, Y104I, S156E, N163R, A194P, and A215K. Preferably, the variant comprises or consists of SEQ ID NO:79.

In one aspect, the variant comprises or consists of SEQ ID NO:3 with the substitutions S99F, S101L, and N163R. Preferably, the variant comprises or consists of SEQ ID NO:80.

In one aspect, the variant comprises or consists of SEQ ID NO:3 with the substitutions S99F, S101L, S103T, Y104I, and N163R. Preferably, the variant comprises or consists of SEQ ID NO:81.

In one aspect, the variant comprises or consists of SEQ ID NO:3 with the substitutions S99F, S101L, S103T, Y104I, N163R, A194P, and A215K. Preferably, the variant comprises or consists of SEQ ID NO:82.

In one aspect, the variant comprises or consists of SEQ ID NO:3 with the substitutions S99F, S101L, S103T, Y104I, and A215K. Preferably, the variant comprises or consists of SEQ ID NO:83.

In one aspect, the variant comprises or consists of SEQ ID NO:3 with the substitutions S99F, S101L, S156E, N163R, A194P, and A215K. Preferably, the variant comprises or consists of SEQ ID NO:84.

In one aspect, the variant comprises or consists of SEQ ID NO:3 with the substitutions S99F, S101L, S103T, Y104I, N163R, and A215K. Preferably, the variant comprises or consists of SEQ ID NO:85.

In one aspect, the variant comprises or consists of SEQ ID NO:3 with the substitutions S99F, S101L, S103T, Y104I, N163R, and A194P. Preferably, the variant comprises or consists of SEQ ID NO:86.

In one aspect, the variant comprises or consists of SEQ ID NO:4 with the substitutions N99F, R101L, T194P, A215K. Preferably, the variant comprises or consists of SEQ ID NO:87.

In one aspect, the variant comprises or consists of SEQ ID NO:4 with the substitutions N99F, R101L, S103T, and V104I. Preferably, the variant comprises or consists of SEQ ID NO:88.

In one aspect, the variant comprises or consists of SEQ ID NO:4 with the substitutions N99F, R101L, S103T, V104I, and T194P. Preferably, the variant comprises or consists of SEQ ID NO:89.

In one aspect, the variant comprises or consists of SEQ ID NO:4 with the substitutions N99F, R101L, S103T, V104I, and A215K. Preferably, the variant comprises or consists of SEQ ID NO:90.

In one aspect, the variant comprises or consists of SEQ ID NO:4 with the substitutions N99F, R101L, S103T, V104I, N156E, G163R, and T194P. Preferably, the variant comprises or consists of SEQ ID NO:91.

In addition to the substitutions described above, the variants may comprise further substitutions at one or more other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants of the invention may have one or more improved property compared to the parent. The one or more improved property may be selected from the group consisting of catalytic efficiency, catalytic rate, chemical stability, oxidation stability, pH activity, pH stability, polyester degrading activity, polyester specificity, proteolytic stability, solubility, specific activity, stability under storage conditions, substrate binding, substrate cleavage, substrate specificity, substrate stability, surface properties, thermal activity, and thermostability.

In one aspect, the variants of the invention have on par or improved polyester degrading activity, in particular PLA degrading activity, compared to the parent. Polyester degrading activity may be evaluated using the turbidity assay described below.

In one embodiment, the variants of the present invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In a preferred embodiment, the variants of the present invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the variants of the present invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In a preferred embodiment, the variants of the present invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In one embodiment, the variants of the present invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In a preferred embodiment, the variants of the present invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In one embodiment, the variants of the present invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In a preferred embodiment, the variants of the present invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In one aspect, the variants of the present inventions have improved polyester degrading activity and retained protease activity compared to the parent.

In one embodiment, the variants of the invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to of SEQ ID NO:1, and at least 10%, e.g., at least 20%, at least 30%, at least 40%, at least 50%, at least 60%, at least 70%, at least 80%, at least 90%, at least 95%, or at least 100%, of the protease activity of SEQ ID NO:1.

In a preferred embodiment, the protease variants of the invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1, and at least 10%, e.g., at least 20%, at least 30%, at least 40%, at least 50%, at least 60%, at least 70%, at least 80%, at least 90%, at least 95%, or at least 100%, of the protease activity of SEQ ID NO:1.

In one embodiment, the variants of the invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3, and at least 10%, e.g., at least 20%, at least 30%, at least 40%, at least 50%, at least 60%, at least 70%, at least 80%, at least 90%, at least 95%, or at least 100% of the protease activity of SEQ ID NO:3.

In a preferred embodiment, the protease variants of the invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3, and at least 10%, e.g., at least 20%, at least 30%, at least 40%, at least 50%, at least 60%, at least 70%, at least 80%, at least 90%, at least 95%, or at least 100% of the protease activity of SEQ ID NO:3.

In one embodiment, the variants of the invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4, and at least 10%, e.g., at least 20%, at least 30%, at least 40%, at least 50%, at least 60%, at least 70%, at least 80%, at least 90%, at least 95%, or at least 100% of the protease activity of SEQ ID NO:4.

In a preferred embodiment, the protease variants of the invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4, and at least 10%, e.g., at least 20%, at least 30%, at least 40%, at least 50%, at least 60%, at least 70%, at least 80%, at least 90%, at least 95%, or at least 100% of the protease activity of SEQ ID NO:4.

In one embodiment, the variants of the invention have on par or improved polyester degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5, and at least 10%, e.g., at least 20%, at least 30%, at least 40%, at least 50%, at least 60%, at least 70%, at least 80%, at least 90%, at least 95%, or at least 100% of the protease activity of SEQ ID NO:5.

In a preferred embodiment, the protease variants of the invention have on par or improved PLA degrading activity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5, and at least 10%, e.g., at least 20%, at least 30%, at least 40%, at least 50%, at least 60%, at least 70%, at least 80%, at least 90%, at least 95%, or at least 100% of the protease activity of SEQ ID NO:5.

In one aspect, the variants of the invention have on par or improved thermostability compared to the parent. Thermostability may evaluated by differential scanning calorimetry as described below.

In one embodiment, the variants of the present invention have on par or improved thermostability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the variants of the present invention have on par or improved thermostability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In one embodiment, the variants of the present invention have on par or improved thermostability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In one embodiment, the variants of the present invention have on par or improved thermostability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In one aspect, the variants of the invention have on par or improved proteolytic stability compared to the parent.

In one embodiment, the protease variants of the invention have on par or improved proteolytic stability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the protease variants of the invention have on par or improved proteolytic stability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In one embodiment, the protease variants of the invention have on par or improved proteolytic stability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In one embodiment, the protease variants of the invention have on par or improved proteolytic stability, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In one aspect, the variants of the invention have on par or improved polyester specificity, in particular PLA specificity, compared to the parent.

In one embodiment, the protease variants of the invention have on par or improved polyester specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In a preferred embodiment, the protease variants of the invention have on par or improved PLA specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the protease variants of the invention have on par or improved polyester specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In a preferred embodiment, the protease variants of the invention have on par or improved PLA specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In one embodiment, the protease variants of the invention have on par or improved polyester specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In a preferred embodiment, the protease variants of the invention have on par or improved PLA specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In one embodiment, the protease variants of the invention have on par or improved polyester specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In a preferred embodiment, the protease variants of the invention have on par or improved PLA specificity, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

In one aspect, the variants of the invention have on par or improved solubility compared to the parent.

In one embodiment, the protease variants of the invention have on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the protease variants of the invention have on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:3.

In one embodiment, the protease variants of the invention have on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:4.

In one embodiment, the protease variants of the invention have on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

### Parent Proteases

Protease variants of the invention may be based on any parent protease. The parent may be a naturally occurring (wild-type) polypeptide or a variant or fragment thereof.

In one aspect, the parent protease has a sequence identity to the polypeptide of SEQ ID NO:1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to 20 amino acids, e.g., 1, 2, 3, 4, 5, 6, 7, 8, 9, 10, 11, 12, 13, 14, 15, 16, 17, 18, 19, or 20, from the polypeptide of SEQ ID NO:1. In an embodiment, the parent comprises, consists essentially of, or consists of the amino acid sequence of SEQ ID NO:1.

In one aspect, the parent protease has a sequence identity to the polypeptide of SEQ ID NO:3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to 20 amino acids, e.g., 1, 2, 3, 4, 5, 6, 7, 8, 9, 10, 11, 12, 13, 14, 15, 16, 17, 18, 19, or 20, from the polypeptide of SEQ ID NO:3. In an embodiment, the parent comprises, consists essentially of, or consists of the amino acid sequence of SEQ ID NO:3.

In one aspect, the parent protease has a sequence identity to the polypeptide of SEQ ID NO:4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and has protease activity. In an embodiment aspect, the amino acid sequence of the parent differs by up to 20 amino acids, e.g., 1, 2, 3, 4, 5, 6, 7, 8, 9, 10, 11, 12, 13, 14, 15, 16, 17, 18, 19, or 20, from the polypeptide of SEQ ID NO:4. In an embodiment, the parent comprises, consists essentially of, or consists of the amino acid sequence of SEQ ID NO:4.

Some variants of the invention comprise the substitutions S9E, N43R, N76D, V205I, Q206L, Y209W, S259D, N261W, and L262E. The polypeptide consisting of SEQ ID NO:1 with the substitutions S9E, N43R, N76D, V205I, Q206L, Y209W, S259D, N261W, and L262E is identical to SEQ ID NO:5, which is a stabilized variant of SEQ ID NO:1. Thus, protease variants of the invention that are variants of SEQ ID NO:1 and comprise inter alia the substitutions S9E, N43R, N76D, V205I, Q206L, Y209W, S259D, N261W, and L262E may also be perceived as variants of SEQ ID NO:5.

In an aspect, the parent protease has a sequence identity to the polypeptide of SEQ ID NO:5 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and has protease activity. In an embodiment, the amino acid sequence of the parent differs by up to 20 amino acids, e.g., 1, 2, 3, 4, 5, 6, 7, 8, 9, 10, 11, 12, 13, 14, 15, 16, 17, 18, 19, or 20, from the polypeptide of SEQ ID NO:5. In an embodiment, the parent comprises, consists essentially of, or consists of the amino acid sequence of SEQ ID NO:5.

The parent polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial protease. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis protease.

In another aspect, the parent is a Streptococcus equisimilis, Streptococcus pyogenes, Streptococcus uberis, or Streptococcus equi subsp. Zooepidemicus protease.

In another aspect, the parent is a Streptomyces achromogenes, Streptomyces avermitilis, Streptomyces coelicolor, Streptomyces griseus, or Streptomyces lividans protease.

In another aspect, the parent is SEQ ID NO:1.

In another aspect, the parent is SEQ ID NO:3.

In another aspect, the parent is SEQ ID NO:4.

In another aspect, the parent is SEQ ID NO:5.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989).

### Preparation of Protease Variants

The present invention also relates to methods for obtaining a protease variant having polyester degrading activity, comprising: (a) introducing into a parent protease the substitutions X99F and X101L, and introducing at least one substitution selected from the group consisting of X3T, X97D, X103T, X104I, X127I, X161W, X161R, X161Y, X161L, X161V, X163R, X172K, X174G, X174V, X175A, X175Y, X175D, X175T, X175V, X175I, X176S, X194P, and X215K; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding a variant of the present invention.

The techniques used to isolate or clone a polynucleotide are known in the art and include isolation from genomic DNA or cDNA, or a combination thereof. The cloning of the polynucleotides from genomic DNA can be achieved, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a non-translated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus nigeralpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alphaamylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Preferably, the bacterial host cell is a Bacillus licheniformis cell.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be achieved by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be achieved by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be achieved by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be achieved by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be achieved by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising (a) cultivating a recombinant host cell of the present invention under conditions conducive for production of the variant; and optionally (b) recovering the variant.

The recombinant host cells are cultivated in a nutrient medium suitable for production of the variant using methods known in the art. For example, the cells may be cultivated by shake flask cultivation, or small-scale or large-scale fermentation (including continuous, batch, fedbatch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Fermentation Broth Formulations or Cell Compositions

The present invention also relates to a fermentation broth formulation or a cell composition comprising a variant of the present invention. The fermentation broth product further comprises additional ingredients used in the fermentation process, such as, for example, cells (including, the host cells containing the gene encoding the variant of the present invention which are used to produce the variant of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Compositions

The present invention also relates to compositions comprising a protease variant or a host cell of the invention. In the context of the invention, the term “composition” encompasses any kind of compositions comprising a variant of the invention. In a particular embodiment, the protease variant is in isolated or at least partially purified form.

The composition may be liquid or dry, for instance in the form of a powder. In some embodiments, the composition is a lyophilisate. For instance, the composition may comprise the protease variant sand/or host cells encoding the variant of the invention or an extract thereof containing said protease variant, and optionally excipients and/or reagents, etc. Appropriate excipients encompass buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g. sorbitol, trehalose or lactose, glycerol, polyethyleneglycol, polyethene glycol, polypropylene glycol, propylene glycol, divalent ions such as calcium, sequestering agent such as EDTA, reducing agents, amino acids, a carrier such as a solvent or an aqueous solution, and the like. The composition of the invention may be obtained by mixing the protease variant with one or several excipients

The composition of the invention may comprise from 0.1% to 99.9%, preferably from 0.1% to 50%, more preferably from 0.1% to 30%, even more preferably from 0.1% to 5% by weight of the variant of the invention and from 0.1% to 99.9%, preferably from 50% to 99.9%, more preferably from 70% to 99.9%, even more preferably from 95% to 99.9% by weight of excipient(s). A preferred composition comprises between 0.1 and 5% by weight of the variant of the invention. In another embodiment, the composition of the invention comprises from 0.1% to 40%, more preferably from 1% to 30%, even more preferably from 5% to 25% by weight of the variant of the invention and from 60% to 99.9%, preferably from 70% to 99%, more preferably from 75% to 95% by weight of excipient(s).

In a particular embodiment, the composition may further comprise additional polypeptide(s) exhibiting an enzymatic activity. The amounts of protease variant of the invention will be easily adapted by those skilled in the art depending, e.g., on the nature of the polyester containing material to degrade and/or the additional enzymes/polypeptides contained in the composition. In one embodiment, the composition may comprise a variant of the present invention as the major enzymatic component, e.g., a mono-component composition. Alternatively, the compositions may comprise multiple enzymatic activities, such as one or more enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

In a particular embodiment, the protease variant of the invention is solubilized in an aqueous medium together with one or several excipients, especially excipients which are able to stabilize or protect the polypeptide from degradation. For instance, the protease variant of the invention may be solubilized in water, eventually with additional components, such as glycerol, sorbitol, dextrin, starch, glycol such as propanediol, salt, etc. The resulting mixture may then be dried so as to obtain a powder. Methods for drying such mixture are well-known in the art and include, without limitation, lyophilization, freeze-drying, spray-drying, supercritical drying, downdraught evaporation, thin-layer evaporation, centrifugal evaporation, conveyer drying, fluidized bed drying, drum drying or any combination thereof.

In a further particular embodiment, the composition of the invention comprises at least one host cell expressing a protease variant of the invention, or an extract thereof. An “extract of a cell” designates any fraction obtained from a cell, such as cell supernatant, cell debris, cell walls, DNA extract, enzymes or enzyme preparation or any preparation derived from cells by chemical, physical and/or enzymatic treatment, which is essentially free of living cells. Preferred extracts are enzymatically active extracts. The composition of the invention may comprise one or several host cells of the invention or extract thereof containing a protease variant of the invention, and optionally one or several additional cells.

In a particular embodiment, the composition comprises or consists of a lyophilized culture medium of a recombinant microorganism expressing a protease variant of the invention. In a particular embodiment, the powder comprises the protease variant of the invention and a stabilizing/solubilizing amount of glycerol, sorbitol or dextrin, such as maltodextrin and/or cyclodextrin, starch, arabic gum, glycol such as propanediol, and/or salt.

The protease variants of the invention are particularly useful in biodegradable plastic products containing said variant and at least one. Thus, the present invention also relates to a composition, such as a plastic material or product, comprising a) a protease variant of the invention; and b) at least one polyester. In an embodiment, the at least one polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly(ethylene adipate) (PEA) and blends/mixtures of these materials. Preferably, the at least one polyester is PLA. In an embodiment, the composition further comprises one or more additional enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

### Use of Protease Variants

The present invention also provides methods using a protease variant of the invention for degrading and/or recycling polyesters and polyester containing materials such as plastic products made of or containing polyesters. The present invention also provides methods for producing biodegradable plastic materials or products comprising a protease variant of the invention and a polyester.

In an aspect, the present invention relates to use of a protease variant of the invention for degrading a polyester containing material, such as a PLA-containing material.

In an aspect, the present invention relates to a method for degrading a polyester containing material (e.g., a plastic product containing at least one polyester), wherein the material is contacted with a protease variant of the invention, whereby the material is degraded. Preferably, the polyester(s) of the polyester containing material may be degraded to monomers and/or oligomers. In an embodiment, the polyester is degraded to yield re-polymerizable monomers and/or oligomers, which are advantageously retrieved in order to be used. In a preferred embodiment, the polyester is PLA and is degraded to yield re-polymerizable monomers and/or oligomers of lactic acid (LA). In an embodiment, the polyester is fully degraded. In a particular embodiment, the polyester containing material comprises at least one polyester selected from polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly(ethylene adipate) (PEA), and blends/mixtures of these materials; preferably polylactic acid (PLA).

In an aspect, the present invention also relates to a method of producing monomers and/or oligomers from a polyester containing material, the method comprising (a) exposing a polyester containing material to a protease variant of the invention; and optionally (b) recovering the monomers and/or oligomers. The method of the invention is particularly useful for producing lactic acid monomers from plastic products containing PLA.

The time required for degrading a polyester containing material may vary depending on the polyester containing material itself (i.e., nature and origin of the material, its composition, shape, etc.), the type and amount of protease variant used, as well as various process parameters (i.e., temperature, pH, additional agents, etc.). One skilled in the art may easily adapt the process parameters to the polyester containing material.

Advantageously, the degradation process is implemented at a temperature between 20° C. and 90° C., preferably between 40° C. and 80° C., more preferably between 60° C. and 80° C., more preferably between 70° C. and 80° C., even more preferably at 75° C. More generally, the temperature is maintained below an inactivating temperature, which corresponds to the temperature at which the protease variant is inactivated. Preferably, the temperature is maintained below the glass transition temperature (Tg) of the polyester in the polyester containing material. In this embodiment, the degradation process is implemented at a temperature comprised between 20° C. and 80° C., preferably between 30° C. and 70° C., more preferably between 40° C. and 60° C., more preferably between 40° C. and 50° C., even more preferably at 45° C. More particularly, the process may be implemented in a continuous way, at a temperature at which the protease variant can be used several times and/or recycled.

Advantageously, the degradation process is implemented at a pH value between 5 and 11, preferably at a pH value between 6 and 10, more preferably at a pH value between 6.5 and 9, even more preferably at a pH value between 7 and 8.

In a particular embodiment, the polyester containing material may be pretreated prior to be contacted with the protease variant in order to physically change its structure, so as to increase the surface of contact between the polyester and the variant. Optionally, monomers and/or oligomers resulting from the depolymerization may be recovered, sequentially or continuously. A single type of monomers and/or oligomers or several different types of monomers and/or oligomers may be recovered, depending on the starting polyester containing material.

The recovered monomers and/or oligomers may be further purified, using all suitable purifying methods and conditioned in a re-polymerizable form. Examples of purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, and combinations hereof.

The repolymerizable monomers and/or oligomers may then be used for instance to synthesize polyesters. Advantageously, polyesters of same nature are repolymerized. However, it is possible to mix the recovered monomers and/or oligomers with other monomers and/or oligomers, in order for instance to synthesize new copolymers. Alternatively, the recovered monomers may be used as chemical intermediates in order to produce new chemical compounds of interest.

In one aspect, the present invention provides a polyester containing material comprising at least one polyester and a protease variant of the invention. In an embodiment, the polyester containing material comprises at least one polyester selected from polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly(ethylene adipate) (PEA), and blends/mixtures of these materials. Preferably the at least one polyester is polylactic acid (PLA). In a particular embodiment, such polyester containing material may be a plastic compound, a masterbatch composition, and/or a plastic product. In the context of the invention, a “masterbatch composition” refers to a concentrated mixture of selected ingredients (e.g., active agents, additives, etc.) that can be used for introducing said ingredients into plastic compounds or products in order to impart desired properties. Masterbatch compositions may be solid or liquid. Preferably, masterbatch compositions of the invention contain at least 10% by weight of active ingredients, more preferably of protease variant of the invention.

In one aspect, the present invention relates to a plastic compound containing a protease variant of the invention and at least one polyester. In a particular embodiment, the polyester is polylactic acid (PLA), preferably poly(L-lactic acid) (PLLA), poly(D-lactic acid) (PDLA) or poly(DL-lactic acid) (PDLLA). In a particular embodiment, the plastic compound may contain an additional polymer, preferably selected from polyesters such as PEAT, PCL, PET; polyolefins such as polyethylene, polypropylene; or natural polymers such as starch, cellulose or flour; and blends/mixtures thereof. More particularly, the plastic compound may contain additional polymers selected from PEAT, flour or starch. In another particular embodiment, the polyester is preferably polycaprolactone (PCL).

In one aspect, the present invention relates to a masterbatch composition containing a protease variant of the invention and at least one polyester. In a particular embodiment, the polyester is polylactic acid (PLA), preferably poly(L-lactic acid) (PLLA), poly(D-lactic acid) (PDLA) or poly(DL-lactic acid) (PDLLA). In another particular embodiment, the polyester is preferably polycaprolactone (PCL).

The present invention also relates to a process for producing polyester containing materials (i.e., plastic compounds, masterbatch compositions, or plastic products) comprising mixing a polyester and a protease variant at a temperature at which the polyester is in a partially or totally molten state, wherein the protease variant is included in the polyester containing material. In a particular embodiment, the process is an extrusion process. For instance, the protease variant and the polyester may be mixed at a temperature between the glass transition temperature and the melting point of the polyester. Alternatively, the protease variant and the polyester may be mixed at a temperature corresponding to the melting point of said polyester, or above. In a particular embodiment, the protease variant and the polyester are mixed at a temperature between 40° C. and 250° C., preferably between 50° C. and 180° C. Alternatively, the protease variant and the polyester are mixed at a temperature above 40° C., preferably above 50° C., even more preferably above 60° C.

In a preferred embodiment, the polyester is polylactic acid (PLA), and the protease variant and PLA are mixed at a temperature between 60° C. and 250° C., preferably between 100° C. and 200° C., more preferably between 130° C. and 180° C., even more preferably between 140° C. and 160° C. Alternatively, the protease variant and PLA are mixed at a temperature above 80° C., preferably above 100° C., even more preferably above 130° C., and below 180° C.,

In another preferred embodiment, the polyester is polycaprolactone (PCL) and the protease variant and PCL are mixed at a temperature between 40° C. and 100° C., preferably between 50° C. and 80° C. Alternatively, the protease variant and PCL are mixed at a temperature above 40° C., preferably above 50° C., even more preferably above 55° C., and below 80° C.

More preferably, the mixing step is performed using extrusion, twin screw extrusion, single screw extrusion, injection-molding, casting, thermoforming, rotary molding, compression, calendering, ironing, coating, stratification, expansion, pultrusion, extrusion blow-molding, extrusion-swelling, compression-granulation, water-in-oil-in-water double emulsion evaporation, 3D printing or similar techniques known by person skilled in the art.

The resulting plastic compound, masterbatch composition, or plastic product comprises a protease variant of the invention embedded in the mass of the plastic compound, masterbatch composition, or plastic product. Advantageously, such plastic compound or masterbatch composition can be used in the production of polyester containing materials and/or plastic products that will thus include a protease variant of the invention.

In a particular embodiment, the resulting plastic compound, masterbatch composition, or plastic product is a biodegradable plastic compound, masterbatch composition, or plastic product complying with at least one of the relevant standards and/or labels known by the person skilled in the art, such as standard EN 13432, standard ASTM D6400, OK Biodegradation Soil (Label Vincotte), OK Biodegradation Water (Label Vincotte), OK Compost (Label Vincotte), or OK Home Compost (Label Vincotte).

### Detergent Compositions

In an aspect, the present invention also relates to a detergent or cleaning composition comprising a protease variant of the invention.

In an embodiment, the detergent or composition comprises a protease variant of the invention and further comprises one or more detergent components and/or one or more additional enzymes. In a preferred embodiment, the detergent composition comprises one or more detergent components, in particular one or more non-naturally occurring detergent components.

The present invention also relates to a detergent composition comprising a protease variant of the present invention and further comprising one or more additional enzymes selected from the group consisting of amylases, catalases, cellulases (e.g., endoglucanases), cutinases, haloperoxygenases, lipases, mannanases, pectinases, pectin lyases, peroxidases, proteases, xanthanases, lichenases and xyloglucanases, or any mixture thereof.

A detergent composition may, e.g., be in the form of a bar, a homogeneous tablet, a tablet having two or more layers, a pouch having one or more compartments, a regular or compact powder, a granule, a paste, a gel, or a regular, compact or concentrated liquid. In a preferred embodiment, the detergent composition is in the form of a liquid or gel, in particular a liquid laundry detergent.

The invention also relates to use of a detergent composition of the present in a cleaning process, such as laundry or hard surface cleaning such as dish wash, e.g., automated dish washing.

In one embodiment, the protease variant is a variant having a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to the parent polypeptide of SEQ ID NO:1; wherein the variant comprises the substitution Y209W and at least two substitutions selected from the group consisting of S9E, N43R, N76D, V205I, Q206L, S259D, N261W, and L262E; wherein the variant comprises at least one substitution selected from the group consisting of S3T, G97D, S99F, S101L, S103T, V104I, G127I, S156E, S161W, S161R, S161Y, S161L, S161V, S163R, A172K, A174G, A174V, M175A, M175Y, M175D, M175T, M175V, M175I, A176S, A194P, and A215K; wherein the variant has polyester degrading activity and, optionally, protease activity; and wherein position numbers are based on the numbering of SEQ ID NO:2.

In one embodiment, the protease variant is a variant having a sequence identity of at least 60%, e.g., at least 65%, at least 70%, at least 75%, at least 80%, at least 85%, at least 90%, at least 95%, at least 96%, at least 97%, at least 98%, or at least 99%, but less than 100%, to the parent polypeptide of SEQ ID NO:1; wherein the variant comprises the substitution Y209W and at least two substitutions selected from the group consisting of S9E, N43R, N76D, V205I, Q206L, S259D, N261W, and L262E; wherein the variant comprises at least one substitution selected from the group consisting of S3T, G97D, S99F, S101L, S103T, V104I, G127I, S161W, S161R, S161Y, S161L, S161V, A172K, A174G, A174V, M175A, M175Y, M175D, M175T, M175V, M175I, A176S, A194P, and A215K; wherein the variant has polyester degrading activity and, optionally, protease activity; and wherein position numbers are based on the numbering of SEQ ID NO:2.

In a preferred embodiment, the variant comprises or consists of SEQ ID NO:1 with the substitutions S9E, N43R, N76D, V205I, Q206L, Y209W, S259D, N261W, and L262E, and at least one substitution, e.g., at least two, at least three, at least four, at least five, at least six, at least seven, at least eight, at least nine, or at least ten, substitutions selected from the group consisting of S3T, G97D, S99F, S101L, S103T, V104I, G127I, S156E, S161W, S161R, S161Y, S161L, S161V, S163R, A172K, A174G, A174V, M175A, M175Y, M175D, M175T, M175V, M175I, A176S, A194P, and A215K.

In a preferred embodiment, the variant comprises or consists of SEQ ID NO:1 with the substitutions S9E, N43R, N76D, V205I, Q206L, Y209W, S259D, N261W, and L262E, and at least one substitution, e.g., at least two, at least three, at least four, at least five, at least six, at least seven, at least eight, at least nine, or at least ten, substitutions selected from the group consisting of S3T, G97D, S99F, S101L, S103T, V104I, G127I, S161W, S161R, S161Y, S161L, S161V, A172K, A174G, A174V, M175A, M175Y, M175D, M175T, M175V, M175I, A176S, A194P, and A215K.

In a preferred embodiment, the variant comprises or consists of SEQ ID NO:1 with the substitutions S9E, N43R, N76D, V205I, Q206L, Y209W, A215K, S259D, N261W, and L262E.

In one embodiment, the variant has polyester degrading activity and protease activity. In a preferred embodiment, the variant has improved polyester degrading activity and/or improved protease activity compared to SEQ ID NO:1.

In one embodiment, the variant has on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:1.

In one embodiment, the variant has on par or improved solubility, e.g., at least 100%, at least 101%, at least 102%, at least 103%, at least 104%, at least 105%, at least 110%, at least 120%, at least 130%, at least 140%, at least 130%, at least 140%, at least 150%, at least 175%, at least 200%, at least 250%, at least 300%, at least 400%, at least 500%, or more, compared to SEQ ID NO:5.

The choice of additional components for a detergent composition is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product.

In a particular embodiment, a detergent composition comprises a protease variant of the invention and one or more non-naturally occurring detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, antiredeposition agents, enzyme inhibitors or stabilizers, enzyme activators, antioxidants, and solubilizers.

In one embodiment, the protease variant of the invention may be added to a detergent composition in an amount corresponding to 0.01-200 mg of enzyme protein per liter of wash liquor, preferably 0.05-50 mg of enzyme protein per liter of wash liquor, in particular 0.1-10 mg of enzyme protein per liter of wash liquor.

An automatic dish wash (ADW) composition may for example include 0.001%-30%, such as 0.01%-20%, such as 0.1-15%, such as 0.5-10% of enzyme protein by weight of the composition.

A granulated composition for laundry may for example include 0.001%-20%, such as 0.01%-10%, such as 0.05%-5% of enzyme protein by weight of the composition.

A liquid composition for laundry may for example include 0.0001%-10%, such as 0.001-7%, such as 0.1%-5% of enzyme protein by weight of the composition.

The enzymes such as the protease variant of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO 1992/19709 and WO 1992/19708 or the variants according to the invention may be stabilized using peptide aldehydes or ketones such as described in WO 2005/105826 and WO 2009/118375.

The protease variants of the invention may be formulated in liquid laundry compositions such as a liquid laundry compositions composition comprising:
a) at least 0.01 mg of active protease variant per liter detergent,
b) 2 wt % to 60 wt % of at least one surfactant
c) 5 wt % to 50 wt % of at least one builder

The detergent composition may be formulated into a granular detergent for laundry. Such detergent may comprise;
a) at least 0.01 mg of active protease variant per gram of composition
b) anionic surfactant, preferably 5 wt % to 50 wt %
c) nonionic surfactant, preferably 1 wt % to 8 wt %
d) builder, preferably 5 wt % to 40 wt %, such as carbonates, zeolites, phosphate builder, calcium sequestering builders or complexing agents.

Although components mentioned below are categorized by general header according to a particular functionality, this is not to be construed as a limitation, as a component may comprise additional functionalities as will be appreciated by the person skilled in the art.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized. Surfactants lower the surface tension in the detergent, which allows the stain being cleaned to be lifted and dispersed and then washed away.

When included therein, the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein, the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein, the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N(tallow-alkyl)-N,N-bis(2-hydroxyethyl)amine oxide, fatty acid alkanolamides and ethoxylated fatty acid alkanolamides, and combinations thereof.

When included therein, the detergent will usually contain from about 0% to about 10% by weight of a zwitterionic surfactant. Non-limiting examples of zwitterionic surfactants include betaine, alkyldimethylbetaine, sulfobetaine, and combinations thereof.

### Builders and Co-Builders

The detergent composition may contain about 0-65% by weight, such as about 5% to about 45% of a detergent builder or co-builder, or a mixture thereof. In a dish wash detergent, the level of builder is typically 40-65%, particularly 50-65%. Builders and chelators soften, e.g., the wash water by removing the metal ions form the liquid. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-20% by weight, such as about 5% to about 10%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N, N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis (methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEG L), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854 and U.S. Pat. No. 5,977,053.

The protease variants of the invention may also be formulated into a dish wash composition, preferably an automatic dish wash composition (ADVV), comprising:
a) at least 0.01 mg of active protease variant according to the invention, and
b) 10-50 wt % builder preferably selected from citric acid, methylglycine-N,N-diacetic acid (MGDA) and/or glutamic acid-N,N-diacetic acid (GLDA) and mixtures thereof, and
c) at least one bleach component.

### Bleaching Systems

The detergent may contain 0-50% by weight, such as about 0.1% to about 25%, of a bleaching system. Bleach systems remove discolor often by oxidation, and many bleaches also have strong bactericidal properties, and are used for disinfecting and sterilizing. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin have good hydrolytic stability in the product upon storage and are efficient bleach activators. Finally, ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst or a booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)₂, and [2,2′,2″-nitrilotris(ethane-1,2-diylazanylylidene-κN-methanylylidene)triphenolato-κ3O]manganese(III). The bleach catalysts may also be other metal compounds, such as iron or cobalt complexes.

In some embodiments, the bleach component may be an organic catalyst selected from the group consisting of organic catalysts having the following formula:

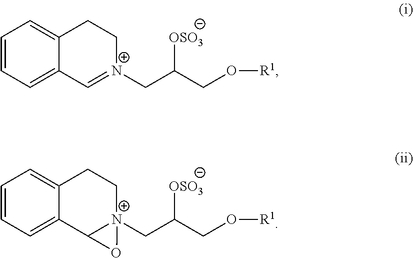

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, n-dodecyl, ntetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

### Hydrotropes

A hydrotrope is a compound that solubilizes hydrophobic compounds in aqueous solutions (or oppositely, polar substances in a non-polar environment). Typically, hydrotropes have both hydrophilic and hydrophobic characters (so-called amphiphilic properties as known from surfactants); however, the molecular structures of hydrotropes generally do not favour spontaneous self-aggregation, see, e.g., review by Hodgdon and Kaler, 2007, Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behaviour, stability, and colloidal properties of systems containing substances of polar and non-polar character, including mixtures of water, oil, surfactants, and polymers. Hydrotropes are classically used across industries from pharma, personal care and food to technical applications. Use of hydrotropes in detergent compositions allows for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium ptoluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt. % to about 0.2 wt. %, from about 0.00008 wt. % to about 0.05 wt. %, or even from about 0.0001 wt. % to about 0.04 wt. % fabric hueing agent. The composition may comprise from 0.0001 wt % to 0.2 wt. % fabric hueing agent, this may be especially preferred when the composition is in the form of a unit dose pouch. Suitable hueing agents are also disclosed in, e.g., WO 2007/087257 and WO 2007/087243.

### Additional Enzymes

The detergent composition may comprise one or more additional enzymes such as an amylase, an arabinase, a carbohydrase, a cellulase (e.g., endoglucanase), a cutinase, a deoxyribonuclease, a galactanase, a haloperoxygenase, a lipase, a mannanase, an oxidase, e.g., a laccase and/or peroxidase, a pectinase, a pectin lyase, an additional protease, a xylanase, a xanthanase, a xyloglucanase or an oxidoreductase.

When the composition comprises one or more additional enzymes, the additional enzyme is preferably an amylase and/or a lipase, in particular an amylase.

The properties of the selected enzyme(s) should be compatible with the selected detergent (e.g., pH-optimum, compatibility with other enzymatic and non-enzymatic ingredients, etc.).

### Proteases

The composition may, in addition to a protease variant of the invention, comprise one or more additional proteases including those of bacterial, fungal, plant, viral or animal origin. Proteases of microbial origin are preferred. The protease may be an alkaline protease, such as a serine protease or a metalloprotease. A serine protease may for example be of the S1 family, such as trypsin, or the S8 family such as subtilisin. A metalloprotease may for example be a thermolysin from, e.g., family M4 or another metalloprotease such as those from M5, M7 or M8 families.

Examples of metalloproteases are the neutral metalloproteases as described in WO 2007/044993 (Genencor Int.) such as those derived from Bacillus amyloliquefaciens.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase®, Esperase®, Progress® Uno and Progress® Excel (Novozymes A/S), those sold under the tradenames Maxatase®, Maxacal®, Maxapem®, Purafect®, Purafect® Ox, Purafect® OxP, Purafect Prime®, Puramax®, FN2®, FN3®, FN4®, Excellase®, Excellenz P1000™, Excellenz P1250™, Eraser®, Preferenz® P100, Preferenz® P110, Effectenz P1000™, Effectenz P1050™, Effectenz P2000™, Purafast®, Properase®, Opticlean® and Optimase® (Danisco/DuPont), Axapem™ (Gist-Brocades N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase, in particular the S54V variant used in the commercial product Gentle Power Bleach from Huntsman Textile Effects Pte Ltd (WO 2010/100028).

### Amylases

Suitable amylases which can be used together with the protease variant of the invention may be an alpha-amylase or a glucoamylase and may be of bacterial or fungal origin. Chemically modified or protein engineered mutants are included. Amylases include, for example, alphaamylases obtained from Bacillus, e.g., a special strain of Bacillus licheniformis, described in more detail in GB 1,296,839.

Suitable amylases include amylases having SEQ ID NO:2 in WO 95/10603 or variants having 90% sequence identity to SEQ ID NO:3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO:6 in WO 2002/10355 or variants thereof having 90% sequence identity to SEQ ID NO:6. Preferred variants of SEQ ID NO:6 are those having a deletion in positions 181 and 182 and a substitution in position 193.

Other amylases which are suitable are hybrid alpha-amylases comprising residues 1-33 of the alpha-amylase derived from B. amyloliquefaciens shown in SEQ ID NO:6 of WO 2006/066594 and residues 36-483 of the B. licheniformis alpha-amylase shown in SEQ ID NO:4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO:6 of WO 2006/066594 and residues 36-483 of SEQ ID NO: 4 are those having the substitutions:
M197T;
H156Y+A181T+N190F+A209V+Q264S; or
G48A+T491+G107A+H156Y+A181T+N190F+I201F+A209V+Q264S.

Other suitable amylases are amylases having the sequence of SEQ ID NO:6 in WO 99/19467 or variants thereof having 90% sequence identity to SEQ ID NO:6. Preferred variants of SEQ ID NO:6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO:1, SEQ ID NO:3, SEQ ID NO:2 or SEQ ID NO:7 of WO 96/23873 or variants thereof having 90% sequence identity to SEQ ID NO:1, SEQ ID NO:2, SEQ ID NO:3 or SEQ ID NO:7. Preferred variants of SEQ ID NO:1, SEQ ID NO:2, SEQ ID NO:3 or SEQ ID NO:7 are those having a substitution, a deletion or an insertion in one or more of the following positions: 140, 181, 182, 183, 184, 195, 206, 212, 243, 260, 269, 304 and 476, using SEQ ID NO:2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO:1, SEQ ID NO:2 or SEQ ID NO:7 are those having a deletion in positions 183 and 184 and a substitution in one or more of positions 140, 195, 206, 243, 260, 304 and 476.

Other amylases which can be used are amylases having SEQ ID NO:2 of WO 2008/153815, SEQ ID NO:10 in WO 01/66712 or variants thereof having 90% sequence identity to SEQ ID NO:2 of WO 2008/153815 or 90% sequence identity to SEQ ID NO:10 in WO 01/66712. Preferred variants of SEQ ID NO:10 in WO 01/66712 are those having a substitution, a deletion or an insertion in one of more of the following positions: 176, 177, 178, 179, 190, 201, 207, 211 and 264.

Further suitable amylases are amylases having SEQ ID NO:2 of WO 2009/061380 or variants having 90% sequence identity to SEQ ID NO:2 thereof. Preferred variants of SEQ ID NO:2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO:2 are those having the substitution in one of more of the following positions: Q87E, R, Q98R, S125A, N128C, T131I, T165I, K178L, T182G, M201L, F202Y, N225E, R, N272E, R, S243Q, A, E, D, Y305R, R309A, Q320R, Q359E, K444E and G475K and/or deletion in position R180 and/or S181 or of T182 and/or G183. Most preferred amylase variants of SEQ ID NO:2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165I+K178L+T182G+Y305R+G475K,
wherein the variants are C-terminally truncated and optionally further comprise a substitution at position 243 and/or a deletion at position 180 and/or position 181.

Further suitable amylases are amylases having SEQ ID NO:1 of WO 2013/184577 or variants having 90% sequence identity to SEQ ID NO:1 thereof. Preferred variants of SEQ ID NO:1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO:1 are those having the substitution in one of more of the following positions: K176L, E187P, N192FYH, M199L, 1203YF, S241QADN, R458N, T459S, D460T, G476K and G477K and/or a deletion in position R178 and/or S179 or of T180 and/or G181. Most preferred amylase variants of SEQ ID NO:1 comprise the substitutions:
E187P+1203Y+G476K
E187P+1203Y+R458N+T459S+D460T+G476K
and optionally further comprise a substitution at position 241 and/or a deletion at position 178 and/or position 179.

Further suitable amylases are amylases having SEQ ID NO:1 of WO 2010/104675 or variants having 90% sequence identity to SEQ ID NO:1 thereof. Preferred variants of SEQ ID NO:1 are those having a substitution, a deletion or an insertion in one of more of the following positions: N21, D97, V128 K177, R179, S180, I181, G182, M200, L204, E242, G477 and G478.

More preferred variants of SEQ ID NO:1 are those having the substitution in one of more of the following positions: N21D, D97N, V128I K177L, M200L, L204YF, E242QA, G477K and G478K and/or a deletion in position R179 and/or S180 or of I181 and/or G182. Most preferred amylase variants of SEQ ID NO:1 comprise the substitutions N21D+D97N+V128I, and optionally further comprise a substitution at position 200 and/or a deletion at position 180 and/or position 181.

Other suitable amylases are the alpha-amylase having SEQ ID NO:12 in WO 01/66712 or a variant having at least 90% sequence identity to SEQ ID NO:12. Preferred amylase variants are those having a substitution, a deletion or an insertion in one of more of the following positions of SEQ ID NO:12 in WO 01/66712: R28, R118, N174, R181, G182, D183, G184, G186, W189, N195, M202, Y298, N299, K302, S303, N306, R310, N314, R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087. Commercially available amylases include DuramylT™, Termamyl™, Fungamyl™, Stainzyme™, Stainzyme Plus™, Natalase™, Liquozyme X, BAN™, Amplify® and Amplify® Prime (from Novozymes A/S), and Rapidase™, Purastar™/Effectenz™, Powerase, Preferenz S1000, Preferenz S100 and Preferenz S110 (from Genencor International Inc./DuPont).

One preferred amylase is a variant of the amylase having SEQ ID NO:13 in WO 2016/180748 with the alterations H1*+N54S+V56T+K72R+G109A+F113Q+R116Q+W167F+Q172G+A174S+G182*+D183*+G184T+N195F+V206L+K391A+P473R+G476K.

Another preferred amylase is a variant of the amylase having SEQ ID NO:1 in WO 2013/001078 with the alterations D183*+G184*+W140Y+N195F+V206Y+Y243F+E260G+G304R+G476K.

Another preferred amylase is a variant of the amylase having SEQ ID NO:1 in WO 2018/141707 with the alterations H1*+G7A+G109A+W140Y+G182*+D183*+N195F+V206Y+Y243F+E260G+N280S+G304R+E391A+G476K.

A further preferred amylase is a variant of the amylase having SEQ ID NO:1 in WO 2017/191160 with the alterations L202M+T246V.

### Deoxyribonucleases (DNases)

Suitable deoxyribonucleases (DNases) are any enzyme that catalyzes the hydrolytic cleavage of phosphodiester linkages in the DNA backbone, thus degrading DNA. A DNase which is obtainable from a bacterium is preferred, in particular a DNase which is obtainable from a species of Bacillus is preferred; in particular a DNase which is obtainable from Bacillus subtilis or Bacillus licheniformis is preferred. Examples of such DNases are described in WO 2011/098579 and WO 2014/087011.

### Oxidoreductases

In one embodiment, the composition may comprise an oxidoreductase, which are enzymes that catalyze reduction-oxidation reactions. A preferred oxidoreductase is a superoxide dismutase.

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant Science Series, volume 71, Marcel Dekker, Inc., 1997.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0.01% to about 05%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1.,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt. % to upper levels of 0.5 or even 0.75 wt. %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose derivatives such as those described in EP 1867808 or WO 03/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, perfumes, pigments, sod suppressors, solvents, and structurants for liquid detergents and/or structure elasticizing agents.

### Formulation of Detergent Products

The detergent enzyme(s), i.e., a protease variant of the invention and optionally one or more additional enzymes, may be included in a detergent composition by adding separate additives containing one or more enzymes, or by adding a combined additive comprising all of these enzymes. A detergent additive comprising one or more enzymes can be formulated, for example, as a granulate, liquid, slurry, etc. Preferred detergent additive formulations include granulates, in particular non-dusting granulates, liquids, in particular stabilized liquids, or slurries.

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multiple compartments. It can be of any form, shape and material which is suitable for hold the composition, e.g., without allowing the release of the composition from the pouch prior to water contact. The pouch is made from water soluble film which encloses an inner volume. The inner volume can be divided into compartments of the pouch. Preferred films are polymeric materials, preferably polymers which are formed into a film or sheet. Preferred polymers, copolymers or derivates thereof are selected from polyacrylates, and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and hydroxypropyl methyl cellulose (HPMC). Preferably the level of polymer in the film for example PVA is at least about 60%. The preferred average molecular weight will typically be about 20,000 to about 150,000. Films can also be of blend compositions comprising hydrolytically degradable and water-soluble polymer blends such as polylactide and polyvinyl alcohol (known under the Trade reference M8630 as sold by Chris Craft In. Prod. of Gary, Indiana, US) plus plasticizers like glycerol, ethylene glycerol, propylene glycol, sorbitol and mixtures thereof. The pouches can comprise a solid laundry detergent composition or part components and/or a liquid cleaning composition or part components separated by the water-soluble film. The compartment for liquid components can be different in composition than compartments containing solids. See, e.g., US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent which is not unit dosed may be aqueous, typically containing at least 20% by weight and up to 95% water, such as up to about 70% water, up to about 65% water, up to about 55% water, up to about 45% water, or up to about 35% water. Concentrated liquid detergents may have lower water contents, for example not more than about 30% or not more than about 20%, e.g. in the range of about 1% to about 20%, such as from about 2% to about 15%. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

Liquid detergent compositions may be formulated to have a moderate pH of e.g. from about 6 to about 10, such as about pH 7, about pH 8 or about pH 9, or they may be formulated to have a higher pH of e.g. from about 10 to about 12, such as about pH 10, about pH 11 or about pH 12.

Unless indicated otherwise, the term “liquid” as used herein should be understood to encompass any kind of liquid detergent composition, for example concentrated liquids, gels, or the liquid or gel part of e.g. a pouch with one or more compartments.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and thus not a liquid, gel or powder at room temperature.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion, wherein the monovalent cation may be for example Na⁺, K⁺ or NH₄⁺, and the organic anion may be for example formate, acetate, citrate, or lactate such that the salt of a monovalent cation and an organic anion may be, for example, sodium formate.

The laundry soap bar may also contain complexing agents such as EDTA and HEDP, perfumes and/or different type of fillers, surfactants, e.g., anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to mixers, plodders, e.g., a two-stage vacuum plodder, extruders, cutters, logo-stampers, cooling tunnels and wrappers. A premix containing a soap, the enzyme of the invention, optionally one or more additional enzymes, a protease inhibitor, and a salt of a monovalent cation and an organic anion may be prepared, and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known methods, for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

### Uses and Cleaning Methods

The present invention is also directed to methods for using the protease variants according to the invention or compositions thereof in laundering of textile and fabrics, such as household laundry washing and industrial laundry washing.

The invention is also directed to methods for using the variants according to the invention or compositions thereof in cleaning hard surfaces such as floors, tables, walls, roofs etc. as well as surfaces of hard objects such as cars (car wash) and dishes (dish wash).

The protease variants of the present invention may be added to and thus become a component of a detergent composition. Thus, one aspect of the invention relates to the use of a protease variant in a cleaning process such as laundering and/or hard surface cleaning.

A detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

The cleaning process or the textile care process may for example be a laundry process, a dishwashing process or cleaning of hard surfaces such as bathroom tiles, floors, tabletops, drains, sinks and washbasins. Laundry processes can for example be household laundering but may also be industrial laundering. Furthermore, the invention relates to a process for laundering of fabrics and/or garments, where the process comprises treating fabrics with a washing solution containing a detergent composition and at least one protease variant of the invention. The cleaning process or a textile care process can for example be carried out in a machine washing or manually. The washing solution can for example be an aqueous washing solution containing a detergent composition.

The last few years there has been an increasing interest in replacing components in detergents that are derived from petrochemicals with renewable biological components such as enzymes and polypeptides without compromising the wash performance. When the components of detergent compositions change, new enzyme activities or new enzymes having alternative and/or improved properties compared to the previously used detergent enzymes such as proteases, lipases and amylases may be needed to achieve a similar or improved wash performance when compared to the traditional detergent compositions.

The invention further concerns the use of protease variants of the invention in a proteinaceous stain removing process. The proteinaceous stains may be stains such as food stains, e.g., baby food, cocoa, egg or milk, or other stains such as sebum, blood, ink or grass, or a combination hereof.

### Washing Method

The present invention provides a method of cleaning a fabric, dishware or a hard surface with a detergent composition comprising a protease variant of the invention.

The method of cleaning comprises contacting an object with a detergent composition comprising a protease variant of the invention under conditions suitable for cleaning the object. In a preferred embodiment the detergent composition is used in a laundry or a dish wash process.

Another embodiment relates to a method for removing stains from fabric or dishware which comprises contacting the fabric or dishware with a composition comprising a protease of the invention under conditions suitable for cleaning the object. In the method of cleaning of the invention, the object being cleaned may be any suitable object such as a textile or a hard surface such as dishware or a floor, table, wall, etc.

Also contemplated are compositions and methods of treating fabrics (e.g., to desize a textile) using one or more of the protease variants of the invention. The protease can be used in any fabric-treating method which is well known in the art (see, e.g., U.S. Pat. No. 6,077,316). For example, in one aspect, the feel and appearance of a fabric is improved by a method comprising contacting the fabric with a protease in a solution. In one aspect, the fabric is treated with the solution under pressure.

The detergent compositions of the present invention are suited for use in laundry and hard surface applications, including dish wash. Accordingly, the present invention includes a method for laundering a fabric or washing dishware, comprising contacting the fabric/dishware to be cleaned with a solution comprising the detergent composition according to the invention. The fabric may comprise any fabric capable of being laundered in normal consumer use conditions. The dishware may comprise any dishware such as crockery, cutlery, ceramics, plastics such as melamine, metals, china, glass and acrylics. The solution preferably has a pH from about 5.5 to about 11.5. The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

The enzyme(s) of the detergent composition of the invention may be stabilized using conventional stabilizing agents and protease inhibitors, e.g., a polyol such as propylene glycol or glycerol, a sugar or sugar alcohol, different salts such as NaCl; KCl; lactic acid, formic acid, boric acid, or a boric acid derivative, e.g., an aromatic borate ester, or a phenyl boronic acid derivative such as 4-formylphenyl boronic acid, or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and B1 consists of one or more amino acid residues (preferably one, two or three), optionally comprising an N-terminal protection group, or as described in WO 2009/118375, WO 98/13459) ora protease inhibitor of the protein type such as RASI, BASI, WASI (bifunctional alpha-amylase/subtilisin inhibitors of rice, barley and wheat) or Cl2 or SSI. The composition may be formulated as described in, e.g., WO 92/19709, WO 92/19708 and U.S. Pat. No. 6,472,364. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), Nickel (II), and oxovanadium (IV)).

The detergent compositions provided herein are typically formulated such that, during use in aqueous cleaning operations, the wash water has a pH of from about 5.0 to about 12.5, such as from about 5.0 to about 11.5, or from about 6.0 to about 10.5. In some embodiments, granular or liquid laundry products are formulated to have a pH from about 6 to about 8. Techniques for controlling pH at recommended usage levels include the use of buffers, alkalis, acids, etc., and are well known to those skilled in the art.

The present invention is further described by the following examples that should not be construed as limiting the scope of the invention.

### EXAMPLES

### Preparation and Purification of Polypeptides

Mutation and introduction of expression cassettes into Bacillus subtilis was performed by standard methods known in the art. All DNA manipulations were performed by PCR (e.g. as described by Sambrook et al., 2001) using standard methods known to the skilled person. Recombinant B. subtilis constructs encoding protease variants were inoculated into and cultivated in a complex medium (TBgly) under antibiotic selection for 24 h at 37° C. Shake flasks containing a rich media (PS-1: 100 g/L sucrose (Danisco cat. no. 109-0429), 40 g/L crust soy (soybean flour), 10 g/L Na₂HPO₄ 12H₂O (Merck cat. no. 106579), 0.1 ml/L Dowfax63N10 (Dow) were inoculated in a ratio of 1:100 with the overnight culture. Shake flask cultivation was performed for 4 days at 30° C. shaking at 270 rpm.

Purification of culture supernatants was performed as follows: The culture broth is centrifuged at 26000×g for 20 minutes and the supernatant is carefully decanted from the precipitate. The supernatant is filtered through a Nalgene 0.2 μm filtration unit in order to remove the remains of the host cells. The pH in the 0.2 μm filtrate is adjusted to pH 8 with 3 M Tris base and the pH-adjusted filtrate is applied to a MEP Hypercel column (Pall Corporation) equilibrated in 20 mM Tris/HCl, 1 mM CaCl₂), pH 8.0. After washing the column with the equilibration buffer, the column is step-eluted with 20 mM CH3COOH/NaOH, 1 mM CaCl₂), pH 4.5. Fractions from the column are analyzed for protease activity using the Suc-AAPF-pNA assay at pH 9 and peak fractions are pooled. The pH of the pool from the MEP Hypercel column is adjusted to pH 6 with 20% (v/v) CH3COOH or 3 M Tris base and the pH-adjusted pool is diluted with deionized water to the same conductivity as 20 mM MES/NaOH, 2 mM CaCl₂), pH 6.0. The diluted pool is applied to an SP-Sepharose® Fast Flow column (GE Healthcare) equilibrated in 20 mM MES/NaOH, 2 mM CaCl₂), pH 6.0. After washing the column with the equilibration buffer, the protease variant is eluted with a linear NaCl gradient (0→0.5 M) in the same buffer over five column volumes. Fractions from the column are analyzed for protease activity using the Suc-AAPF-pNA assay at pH 9 and active fractions are analyzed by SDS-PAGE. Fractions in which only one band is observed on the Coomassie stained SDS-PAGE gel are pooled as the purified preparation and used for further experiments.

### Protease Activity Assay

Proteolytic activity can be determined by a method employing the Suc-AAPF-pNA substrate. Suc-AAPF-pNA is an abbreviation for N-Succinyl-Alanine-Alanine-Proline-Phenylalanine-p-Nitroanilide, and it is a blocked peptide which can be cleaved by endo-proteases. Following proteolytic cleavage, a free pNA molecule having a yellow color is liberated and can be measured by visible spectrophotometry at wavelength 405 nm. The Suc-AAPF-PNA substrate is manufactured by Bachem (cat. no. L1400, dissolved in DMSO).

The protease sample to be analyzed is diluted in residual activity buffer (100 mM Tris pH 8.6). The assay is performed by transferring 30 μl of diluted enzyme samples to 96 well microtiter plate and adding 70 μl substrate working solution (0.72 mg/ml in 100 mM Tris pH 8.6). The solution is mixed at room temperature and absorption is measured every 20 sec. over 5 minutes at OD 405 nm.

The slope (absorbance per minute) of the time dependent absorption-curve is directly proportional to the activity of the protease in question under the given set of conditions. The protease sample should be diluted to a level where the slope is linear.

### PLA Turbidity Assay

A polylactic acid (PLA) emulsion was prepared by solubilizing 0.45 g of PLA in 15 mL of dichloromethane followed by addition of 90 ml of 0.1 M Tris-HCl buffer, pH 9.0. The mixture was sonicated using a Fisherbrand™ Q700 Sonicator with Probe. The amplitude was set to 100% from start but lowered to 1-10% when approx. 10000 J energy was reached. Final energy level was approx. 17000 J. Dichloromethane was evaporated from the emulsion under agitation in a fume hood. When all solvent was evaporated, bigger PLA agglomerates were removed on a sieve (Mesh 100/width 0.15 mm). The resulting filtered PLA emulsion was stored in a refrigerator (<5° C.) until use.

Direct comparisons of two enzymes were always made on the same emulsion analyzed on the same day.

Before conducting the turbidity assay, the PLA emulsion was diluted to a desired start turbidity level using buffer having a desired pH value, e.g., 0.1 M Tris-HCl buffer, pH 9.0 Turbidity was measured using a turbidimeter Hach model 2100 AN. The turbidimeter was equipped with a 13 mm adaptor kit for handling vials containing 5 mL of suspension. A start level of turbidity of approx. 500 NTU was aimed for. Vials containing 5.0 mL of diluted PLA emulsion was heated to 37° C. The initial turbidity of the solution was measured (Tₛₜ) before addition of enzyme at t=0 min. 100 μL of diluted enzyme was added to the vial, and turbidity was measured as a function of time. The change in turbidity, which is indicative of polyester degrading activity, may be calculated as the difference of the value at start and a given timepoint (t): ΔT(t)=Tₛₜ−T(t).

### Determination of Thermal Denaturation Temperature

Thermal stability of variants of the invention was determined by measuring thermal denaturation temperature, Tm, by differential scanning calorimetry (DSC)

Samples for DSC were prepared from purified samples. Buffer or salt from the liquid from the purification step was removed using Sephadex® G-25 gel filtration columns of type NAP™-5. Known amount of protein, e.g., 0.25 mg, was applied to the column and the samples were spun at 1000×g for 3 min. The eluted enzyme was diluted with buffer to reach concentrations of 0.5 mg protein/mL in 50 mM Acetate pH 6.0+2 mM CaCl₂) or 50 mM Glycine pH 9.0+2 mM CaCl₂).

The thermostability of the prepared samples were determined by DSC using a VPCapillary Differential Scanning calorimeter (MicroCal Inc., Piscataway, NJ, USA). The thermal denaturation temperature, Tm (° C.), was determined as the top of denaturation peak (major endothermic peak) in thermograms (Cp vs. T) obtained after heating the 0.5 mg/ml solutions in buffer at a constant programmed heating rate of 200 K/hr. Sample and reference solutions (approx. 0.2 ml) were loaded into the calorimeter (reference: buffer without enzyme) from storage conditions at 10° C. and thermally pre-equilibrated for 20 minutes at 20° C. prior to DSC scan from 20° C. to 110° C. Tm values were determined at an accuracy of approximately +/−1° C.

### Example 1. Screening of First-Generation Protease Variants Based on SEQ ID NO:1

Six different protease variants based on SEQ ID NO:1 were prepared and purified according to the above described procedure and tested in the above described turbidity assay. Compared to SEQ ID NO:1, these variants contain the following substitutions:

| SEQ ID NO: | Substitutions vs. SEQ ID NO: 1 |
|---|---|
| 5 | S9E, N43R, N76D, V205I, Q206L, Y209W, S259D, |
| | N261W, L262E |
| 6 | S9E, N43R, N76D, S99F, S101L, S103T, V104I, V205I, |
| | Q206L, Y209W, S259D, N261W, L262E |
| 7 | S9E, N43R, N76D, S99F, S101L, S103T, V104I, S163R, |
| | V205I, Q206L, Y209W, S259D, N261W, L262E |
| 8 | S9E, N43R, N76D, S99F, S101L, S103T, V104I, S156E, |
| | S163R, V205I, Q206L, Y209W, S259D, N261W, L262E |
| 9 | S9E, N43R, N76D, S99F, S101L, S156E, S163R, V205I, |
| | Q206L, Y209W, S259D, N261W, L262E |
| 10 | S9E, N43R, N76D, S99F, S101L, V104I, S156E, S163R, |
| | V205I, Q206L, Y209W, S259D, N261W, L262E |
| 11 | S9E, N43R, N76D, S101L, V104I, V205I, Q206L, |
| | Y209W, S259D, N261W, L262E |

All variants were dosed at same level of 50 μg enzyme protein, allowing ΔT(t) to be compared. The starting turbidity (Tₛₜ) of the PLA emulsion was approx. 500 NTU, and thus the maximal ΔT(t), which would be equal to a completely clear solution (0 NTU), is consequently 500 NTU.

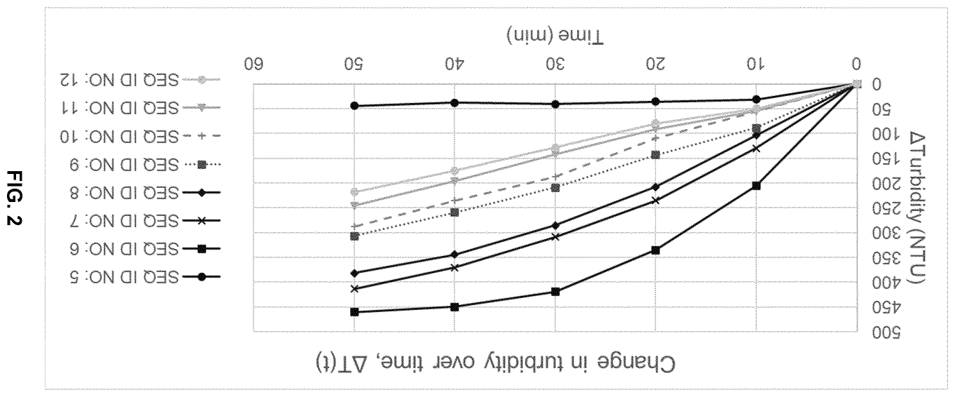

The polyester degrading effect of the SEQ ID NO:5 (a stabilized variant of SEQ ID NO:1, see, e.g., WO 2016/087617) was very low, as evidenced by the flattened curve (see FIG. 2), whereas the other variants showed polyester degrading activity over the entire course of the experiment (50 min of recorded reaction time). Consequently, it is not feasible to calculate an improvement factor compared to SEQ ID NO:5, since this factor will be biased by the time frame selected for calculating ΔT(t) of SEQ ID NO:5. However, by comparing ΔT(50 min) values, an approximated improvement factor can be estimated. Variants SEQ ID NO:6-11 all showed improved polyester degrading activity compared to SEQ ID NO:5, with approximated improvement factors in the range from 4 to 10 (corresponding to 4× and 10× improvements, respectively).

ΔT(t) at different time points

| | ΔT(0 | ΔT(10 | ΔT(20 | ΔT(30 | ΔT(40 | ΔT(50 |
|---|---|---|---|---|---|---|
| SEQ ID NO: | min) | min) | min) | min) | min) | min) |
| 5 | 0 | 32 | 36 | 41 | 39 | 45 |
| 6 | 0 | 206 | 336 | 419 | 450 | 461 |
| 7 | 0 | 131 | 236 | 310 | 371 | 414 |
| 8 | 0 | 104 | 208 | 286 | 345 | 382 |
| 9 | 0 | 56 | 110 | 188 | 236 | 288 |
| 10 | 0 | 55 | 92 | 142 | 197 | 247 |
| 11 | 0 | 51 | 80 | 129 | 176 | 219 |

### Example 2. Screening of Second-Generation Protease Variants Based on SEQ ID NO:6

Based on SEQ ID NO:6, a new variant named SEQ ID NO:12 was prepared and purified according to the above described protocol. Compared to SEQ ID NO:1, SEQ ID NO:12 contains the following substitutions:

| SEQ ID NO: | Substitutions vs. SEQ ID NO: 1 |
|---|---|
| 12 | S9E, N43R, N76D, S99F, S101L, S103T, V104I, V205I, |
| | Q206L, Y209W, A215K, S259D, N261W, L262E |

SEQ ID NO:6 and SEQ ID NO:12 were evaluated in the above described turbidity assay. T(t) was measured and ΔT(t) was calculated for both variants dosed at different levels. ΔT(t) responses are approx. linear as a function of enzyme dosage. Responses of the different dosage levels of SEQ ID NO:12 was directly compared to the same response level of SEQ ID NO:6. The corresponding dosage was calculated using linear regression. Responses after 20 and 30 min were used to calculate an improvement factor as the ratio between the corresponding SEQ ID NO:6 dosage and the actual SEQ ID NO:12 dosage. Compared to SEQ ID NO:6, SEQ ID NO:12 exhibited an averaged improvement factor of 2.0 (see Table below).

ΔT(t) at different time points for SEQ ID NO: 6

| Dose of SEQ | ΔT(0 | ΔT(10 | ΔT(20 | ΔT(30 |
|---|---|---|---|---|
| ID NO: 6 | min) | min) | min) | min) |
| 25 μg | 0 | 102 | 228 | 314 |
| 20 μg | 0 | 90 | 182 | 275 |
| 15 μg | 0 | 72 | 142 | 212 |
| 10 μg | 0 | 53 | 91 | 144 |
| 5 μg | 0 | 36 | 49 | 75 |

ΔT(t) at different time for SEQ ID NO: 12

| Dose of SEQ | ΔT(0 | ΔT(10 | ΔT(20 | ΔT(30 |
|---|---|---|---|---|
| ID NO: 12 | min) | min) | min) | min) |
| 20 μg | 0 | 216 | 343 | 419 |
| 15 μg | 0 | 156 | 280 | 364 |
| 10 μg | 0 | 112 | 215 | 294 |
| 7.5 μg | 0 | 68 | 144 | 199 |
| 5 μg | 0 | 48 | 98 | 145 |

| | Corresponding dose | | | | |
|---|---|---|---|---|---|
| Dose of SEQ | of SEQ ID NO: 6 | | Improvement factor | | |
| ID NO: 12 | 20 min | 30 min | 20 min | 30 min | average |
| 20 μg | 38 μg | 33 μg | 1.9 | 1.6 | 1.8 |
| 15 μg | 31 μg | 28 μg | 2.1 | 1.9 | 2.0 |
| 10 μg | 24 μg | 22 μg | 2.4 | 2.2 | 2.3 |
| 7.5 μg | 16 μg | 15 μg | 2.1 | 1.9 | 2.0 |
| 5 μg | 11 μg | 10 μg | 2.1 | 2.0 | 2.1 |

| | |
|---|---|
| Overall average | 2.0 |

### Example 3. Screening of Third-Generation Protease Variants Based on SEQ ID NO:12

Based on SEQ ID NO:12, a new set of variants were prepared and purified according to the above described protocol and tested in the above described turbidity assay.

SEQ ID NO:12 was evaluated at dosage levels 2.5, 5, 7.5, 10, and 12.5 μg. ΔT(20 min) and ΔT(30 min) curves as a function of dosage was used as standard curves to calculate improvement factors for the variants as described in Example 2. To compare improvement factors directly to SEQ ID NO:6, the improvement factor based on SEQ ID NO:12 as reference is multiplied by two, since the improvement factor of SEQ ID NO:12 compared to SEQ ID NO:6 is 2.0 as estimated in Example 2.

Improvement factors for all third-generation variants are listed in the table below. Selected variants were further evaluated by differential scanning calorimetry for determination of denaturation temperature, Tm, according to procedure described above. Tm values are included in the table below.

| SEQ | Substitutions vs. | Dosage | IF vs SEQ | IF average | Tm (° C.), | Tm (° C.), |
|---|---|---|---|---|---|---|
| ID NO: | SEQ ID NO: 1 | (μg) | ID NO: 6 | (average) | pH = 5 | pH = 9 |
| 6 | S9E, N43R, N76D, S99F, | | 1 (by | 1 (by | 89.4 | 91.2 |
| | S101L, S103T, V104I, | | def.) | def.) | | |
| | V2051, Q206L, Y209W, | | | | | |
| | S259D, N261W, L262E | | | | | |
| 12 | S9E, N43R, N76D, S99F, | | 2.0 | 2.0 | 90.2 | 92.6 |
| | S101L, S103T, V104I, | | | | | |
| | V2051, Q206L, Y209W, | | | | | |
| | A215K, S259D, N261W, | | | | | |
| | L262E | | | | | |
| 13 | S3T, S9E, N43R, N76D, | 5 | 2.12 | 1.98 | 90.8 | 92.7 |
| | S99F, S101L, S103T, | 10 | 1.84 | | | |
| | V104I, A194P, V205I, | | | | | |
| | Q206L, Y209W, A215K, | | | | | |
| | S259D, N261W, L262E | | | | | |
| 14 | S9E, N43R, N76D, S99F, | 7.5 | 1.48 | 1.41 | | |
| | S101L, S103T, V104I, | 10 | 1.34 | | | |
| | S163R, V205I, Q206L, | | | | | |
| | Y209W, A215K, S259D, | | | | | |
| | N261W, L262E | | | | | |
| 15 | S9E, N43R, N76D, S99F, | 5 | 2.60 | 2.53 | | |
| | S101L, S103T, V104I, | 10 | 2.46 | | | |
| | G127I, V205I, Q206L, | | | | | |
| | Y209W, A215K, S259D, | | | | | |
| | N261W, L262E | | | | | |
| 16 | S3T, S9E, N43R, N76D, | 5 | 2.52 | 2.42 | | |
| | S99F, S101L, S103T, | 10 | 2.32 | | | |
| | V104I, G127I, V205I, | | | | | |
| | Q206L, Y209W, A215K, | | | | | |
| | S259D, N261W, L262E | | | | | |
| 17 | S9E, N43R, N76D, S99F, | 7.5 | 1.78 | 1.74 | | |
| | S101L, S103T, V104I, | 10 | 1.70 | | | |
| | G127I, A194P, V205I, | | | | | |
| | Q206L, Y209W, A215K, | | | | | |
| | S259D, N261W, L262E | | | | | |
| 18 | S3T, S9E, N43R, N76D, | 7.5 | 2.02 | 1.94 | | |
| | S99F, S101L, S103T, | 10 | 1.86 | | | |
| | V104I, G127I, A194P, | | | | | |
| | V205I, Q206L, Y209W, | | | | | |
| | A215K, S259D, N261W, | | | | | |
| | L262E | | | | | |
| 19 | S3T, S9E, N43R, N76D, | 6 | 1.18 | 1.18 | | |
| | S99F, S101L, S103T, | | | | | |
| | V104I, A174G, A176S, | | | | | |
| | A194P, V205I, Q206L, | | | | | |
| | Y209W, A215K, S259D, | | | | | |
| | N261W, L262E | | | | | |
| 20 | S3T, S9E, N43R, N76D, | 6 | 1.58 | 1.55 | | |
| | S99F, S101L, S103T, | 10 | 1.52 | | | |
| | V104I, A172K, A174G, | | | | | |
| | A194P, V205I, Q206L, | | | | | |
| | Y209W, A215K, S259D, | | | | | |
| | N261W, L262E | | | | | |
| 21 | S3T, S9E, N43R, N76D, | 10 | 1.56 | 1.56 | | |
| | S99F, S101L, S103T, | | | | | |
| | V104I, S161W, M175A, | | | | | |
| | A194P, V205I, Q206L, | | | | | |
| | Y209W, A215K, S259D, | | | | | |
| | N261W, L262E | | | | | |
| 22 | S3T, S9E, N43R, N76D, | 3 | 2.74 | 2.77 | 77.6 | 80.9 |
| | S99F, S101L, S103T, | 6 | 2.80 | | | |
| | V104I, S161R, M175A, | | | | | |
| | A194P, V205I, Q206L, | | | | | |
| | Y209W, A215K, S259D, | | | | | |
| | N261W, L262E | | | | | |
| 23 | S3T, S9E, N43R, N76D, | 6 | 1.28 | 1.28 | | |
| | S99F, S101L, S103T, | | | | | |
| | V104I, M175A, A176S, | | | | | |
| | A194P, V205I, Q206L, | | | | | |
| | Y209W, A215K, S259D, | | | | | |
| | N261W, L262E | | | | | |
| 24 | S3T, S9E, N43R, N76D, | 10 | 2.54 | 2.54 | | |
| | S99F, S101L, S103T, | | | | | |
| | V104I, A172K, M175A, | | | | | |
| | A194P, V205I, Q206L, | | | | | |
| | Y209W, A215K, S259D, | | | | | |
| | N261W, L262E | | | | | |
| 25 | S3T, S9E, N43R, N76D, | 6 | 1.86 | 1.82 | | |
| | S99F, S101L, S103T, | 10 | 1.78 | | | |
| | V104I, S161Y, M175Y, | | | | | |
| | A194P, V205I, Q206L, | | | | | |
| | Y209W, A215K, S259D, | | | | | |
| | N261W, L262E | | | | | |
| 26 | S3T, S9E, N43R, N76D, | 6 | 1.58 | 1.65 | | |
| | S99F, S101L, S103T, | 10 | 1.72 | | | |
| | V104I, S161L, M175Y, | | | | | |
| | A194P, V205I, Q206L, | | | | | |
| | Y209W, A215K, S259D, | | | | | |
| | N261W, L262E | | | | | |
| 27 | S3T, S9E, N43R, N76D, | 6 | 1.68 | 1.82 | | |
| | S99F, S101L, S103T, | 10 | 1.96 | | | |
| | V104I, M175Y, A176S, | | | | | |
| | A194P, V205I, Q206L, | | | | | |
| | Y209W, A215K, S259D, | | | | | |
| | N261W, L262E | | | | | |
| 28 | S3T, S9E, N43R, N76D, | 10 | 1.36 | 1.36 | | |
| | S99F, S101L, S103T, | | | | | |
| | V104I, A174V, M175Y, | | | | | |
| | A194P, V205I, Q206L, | | | | | |
| | Y209W, A215K, S259D, | | | | | |
| | N261W, L262E | | | | | |
| 29 | S3T, S9E, N43R, N76D, | 6 | 1.18 | 1.35 | | |
| | S99F, S101L, S103T, | 10 | 1.52 | | | |
| | V104I, S161R, M175D, | | | | | |
| | A194P, V205I, Q206L, | | | | | |
| | Y209W, A215K, S259D, | | | | | |
| | N261W, L262E | | | | | |
| 30 | S3T, S9E, N43R, N76D, | 10 | 1.18 | 1.18 | | |
| | S99F, S101L, S103T, | | | | | |
| | V104I, A172K, M175D, | | | | | |
| | A194P, V205I, Q206L, | | | | | |
| | Y209W, A215K, S259D, | | | | | |
| | N261W, L262E | | | | | |
| 31 | S3T, S9E, N43R, N76D, | 2 | 4.34 | 4.93 | | |
| | S99F, S101L, S103T, | 3 | 5.52 | | | |
| | V104I, S161W, M175T, | | | | | |
| | A194P, V205I, Q206L, | | | | | |
| | Y209W, A215K, S259D, | | | | | |
| | N261W, L262E | | | | | |
| 32 | S3T, S9E, N43R, N76D, | 10 | 2.14 | 2.14 | | |
| | S99F, S101L, S103T, | | | | | |
| | V104I, S161V, M175T, | | | | | |
| | A194P, V205I, Q206L, | | | | | |
| | Y209W, A215K, S259D, | | | | | |
| | N261W, L262E | | | | | |
| 33 | S3T, S9E, N43R, N76D, | 10 | 2.00 | 2.00 | | |
| | S99F, S101L, S103T, | | | | | |
| | V104I, S161Y, M175T, | | | | | |
| | A194P, V205I, Q206L, | | | | | |
| | Y209W, A215K, S259D, | | | | | |
| | N261W, L262E | | | | | |
| 34 | S3T, S9E, N43R, N76D, | 1.5 | 5.34 | 5.35 | 80.8 | 88.1 |
| | S99F, S101L, S103T, | 3 | 5.36 | | | |
| | V104I, S161R, M175T, | | | | | |
| | A194P, V205I, Q206L, | | | | | |
| | Y209W, A215K, S259D, | | | | | |
| | N261W, L262E | | | | | |
| 35 | S3T, S9E, N43R, N76D, | 10 | 1.92 | 1.92 | | |
| | S99F, S101L, S103T, | | | | | |
| | V104I, S161L, M175T, | | | | | |
| | A194P, V205I, Q206L, | | | | | |
| | Y209W, A215K, S259D, | | | | | |
| | N261W, L262E | | | | | |
| 36 | S3T, S9E, N43R, N76D, | 10 | 1.62 | 1.62 | | |
| | S99F, S101L, S103T, | | | | | |
| | V104I, M175T, A176S, | | | | | |
| | A194P, V205I, Q206L, | | | | | |
| | Y209W, A215K, S259D, | | | | | |
| | N261W, L262E | | | | | |
| 37 | S3T, S9E, N43R, N76D, | 10 | 1.40 | 1.40 | | |
| | S99F, S101L, S103T, | | | | | |
| | V104I, A174G, M175T, | | | | | |
| | A194P, V205I, Q206L, | | | | | |
| | Y209W, A215K, S259D, | | | | | |
| | N261W, L262E | | | | | |
| 38 | S3T, S9E, N43R, N76D, | 4 | 3.08 | 2.88 | 82.3 | 84.9 |
| | S99F, S101L, S103T, | 7 | 2.68 | | | |
| | V104I, A172K, M175T, | | | | | |
| | A194P, V205I, Q206L, | | | | | |
| | Y209W, A215K, S259D, | | | | | |
| | N261W, L262E | | | | | |
| 39 | S3T, S9E, N43R, N76D, | 10 | 2.16 | 2.16 | | |
| | S99F, S101L, S103T, | | | | | |
| | V104I, S161W, M175V, | | | | | |
| | A194P, V205I, Q206L, | | | | | |
| | Y209W, A215K, S259D, | | | | | |
| | N261W, L262E | | | | | |
| 40 | S3T, S9E, N43R, N76D, | 10 | 2.12 | 2.12 | | |
| | S99F, S101L, S103T, | | | | | |
| | V104I, S161Y, M175V, | | | | | |
| | A194P, V205I, Q206L, | | | | | |
| | Y209W, A215K, S259D, | | | | | |
| | N261W, L262E | | | | | |
| 41 | S3T, S9E, N43R, N76D, | 3 | 4.62 | 4.59 | 85.3 | 88.1 |
| | S99F, S101L, S103T, | 6 | 4.56 | | | |
| | V104I, S161R, M175V, | | | | | |
| | A194P, V205I, Q206L, | | | | | |
| | Y209W, A215K, S259D, | | | | | |
| | N261W, L262E | | | | | |
| 42 | S3T, S9E, N43R, N76D, | 10 | 1.72 | 1.72 | | |
| | S99F, S101L, S103T, | | | | | |
| | V104I, S161L, M175V, | | | | | |
| | A194P, V205I, Q206L, | | | | | |
| | Y209W, A215K, S259D, | | | | | |
| | N261W, L262E | | | | | |
| 43 | S3T, S9E, N43R, N76D, | 10 | 2.44 | 2.44 | | |
| | S99F, S101L, S103T, | | | | | |
| | V104I, M175V, A176S, | | | | | |
| | A194P, V205I, Q206L, | | | | | |
| | Y209W, A215K, S259D, | | | | | |
| | N261W, L262E | | | | | |
| 44 | S3T, S9E, N43R, N76D, | 10 | 1.76 | 1.76 | | |
| | S99F, S101L, S103T, | | | | | |
| | V104I, A174G, M175V, | | | | | |
| | A194P, V205I, Q206L, | | | | | |
| | Y209W, A215K, S259D, | | | | | |
| | N261W, L262E | | | | | |
| 45 | S3T, S9E, N43R, N76D, | 3 | 3.28 | 3.32 | 86.1 | 89.1 |
| | S99F, S101L, S103T, | 6 | 3.36 | | | |
| | V104I, A172K, M175V, | | | | | |
| | A194P, V205I, Q206L, | | | | | |
| | Y209W, A215K, S259D, | | | | | |
| | N261W, L262E | | | | | |
| 46 | S3T, S9E, N43R, N76D, | 10 | 2.40 | 2.40 | | |
| | S99F, S101L, S103T, | | | | | |
| | V104I, S161W, M175I, | | | | | |
| | A194P, V205I, Q206L, | | | | | |
| | Y209W, A215K, S259D, | | | | | |
| | N261W, L262E | | | | | |
| 47 | S3T, S9E, N43R, N76D, | 10 | 2.32 | 2.32 | | |
| | S99F, S101L, S103T, | | | | | |
| | V104I, S161V, M175I, | | | | | |
| | A194P, V205I, Q206L, | | | | | |
| | Y209W, A215K, S259D, | | | | | |
| | N261W, L262E | | | | | |
| 48 | S3T, S9E, N43R, N76D, | 10 | 2.30 | 2.30 | | |
| | S99F, S101L, S103T, | | | | | |
| | V104I, S161Y, M175I, | | | | | |
| | A194P, V205I, Q206L, | | | | | |
| | Y209W, A215K, S259D, | | | | | |
| | N261W, L262E | | | | | |
| 49 | S3T, S9E, N43R, N76D, | 3 | 5.80 | 5.33 | 85.4 | 87.9 |
| | S99F, S101L, S103T, | 6 | 4.86 | | | |
| | V104I, S161R, M175I, | | | | | |
| | A194P, V205I, Q206L, | | | | | |
| | Y209W, A215K, S259D, | | | | | |
| | N261W, L262E | | | | | |
| 50 | S3T, S9E, N43R, N76D, | 10 | 1.96 | 1.96 | | |
| | S99F, S101L, S103T, | | | | | |
| | V104I, S161L, M175I, | | | | | |
| | A194P, V205I, Q206L, | | | | | |
| | Y209W, A215K, S259D, | | | | | |
| | N261W, L262E | | | | | |
| 51 | S3T, S9E, N43R, N76D, | 10 | 1.10 | 1.10 | | |
| | S99F, S101L, S103T, | | | | | |
| | V104I, A174G, M175I, | | | | | |
| | A194P, V205I, Q206L, | | | | | |
| | Y209W, A215K, S259D, | | | | | |
| | N261W, L262E | | | | | |
| 52 | S3T, S9E, N43R, N76D, | 4 | 2.94 | 2.96 | 86.0 | 89.0 |
| | S99F, S101L, S103T, | 7 | 2.98 | | | |
| | V104I, A172K, M175I, | | | | | |
| | A194P, V205I, Q206L, | | | | | |
| | Y209W, A215K, S259D, | | | | | |
| | N261W, L262E | | | | | |
| 53 | S3T, S9E, N43R, N76D, | 10 | 1.96 | 1.96 | | |
| | S99F, S101L, S103T, | | | | | |
| | V104I, S163R, A172K, | | | | | |
| | M175I, A194P, V205I, | | | | | |
| | Q206L, Y209W, A215K, | | | | | |
| | S259D, N261W, L262E | | | | | |
| 54 | S3T, S9E, N43R, N76D, | 10 | 2.26 | 2.26 | | |
| | S99F, S101L, S103T, | | | | | |
| | V104I, S161W, A194P, | | | | | |
| | V205I, Q206L, Y209W, | | | | | |
| | A215K, S259D, N261W, | | | | | |
| | L262E | | | | | |
| 55 | S3T, S9E, N43R, N76D, | 6 | 2.66 | 2.53 | | |
| | S99F, S101L, S103T, | 10 | 2.40 | | | |
| | V104I, S161V, A194P, | | | | | |
| | V205I, Q206L, Y209W, | | | | | |
| | A215K, S259D, N261W, | | | | | |
| | L262E | | | | | |
| 56 | S3T, S9E, N43R, N76D, | 10 | 2.02 | 2.02 | | |
| | S99F, S101L, S103T, | | | | | |
| | V104I, S161Y, A194P, | | | | | |
| | V205I, Q206L, Y209W, | | | | | |
| | A215K, S259D, N261W, | | | | | |
| | L262E | | | | | |
| 57 | S3T, S9E, N43R, N76D, | 2 | 4.58 | 4.81 | 89.4 | 91.3 |
| | S99F, S101L, S103T, | 4 | 5.04 | | | |
| | V104I, S161R, V205I, | | | | | |
| | Q206L, Y209W, A194P, | | | | | |
| | A215K, S259D, N261W, | | | | | |
| | L262E | | | | | |
| 58 | S3T, S9E, N43R, N76D, | 5 | 2.34 | 2.42 | | |
| | S99F, S101L, S103T, | 10 | 2.50 | | | |
| | V104I, M175T, A194P, | | | | | |
| | V205I, Q206L, Y209W, | | | | | |
| | A215K, S259D, N261W, | | | | | |
| | L262E | | | | | |
| 59 | S3T, S9E, N43R, N76D, | 5 | 2.46 | 2.54 | | |
| | S99F, S101L, S103T, | 10 | 2.62 | | | |
| | V104I, M175V, A194P, | | | | | |
| | V205I, Q206L, Y209W, | | | | | |
| | A215K, S259D, N261W, | | | | | |
| | L262E | | | | | |
| 60 | S3T, S9E, N43R, N76D, | 5 | 2.08 | 2.34 | | |
| | S99F, S101L, S103T, | 10 | 2.60 | | | |
| | V104I, M175I, A194P, | | | | | |
| | V205I, Q206L, Y209W, | | | | | |
| | A215K, S259D, N261W, | | | | | |
| | L262E | | | | | |
| 61 | S3T, S9E, N43R, N76D, | 10 | 1.62 | 1.62 | | |
| | S99F, S101L, S103T, | | | | | |
| | V104I, A176S, A194P, | | | | | |
| | V205I, Q206L, Y209W, | | | | | |
| | A215K, S259D, N261W, | | | | | |
| | L262E | | | | | |
| 62 | S3T, S9E, N43R, N76D, | 10 | 1.68 | 1.68 | | |
| | S99F, S101L, S103T | | | | | |
| | V104I, A174G, A194P | | | | | |
| | V205I, Q206L, Y209W, | | | | | |
| | A215K, S259D, N261W, | | | | | |
| | L262E | | | | | |
| 63 | S3T, S9E, N43R, N76D, | 6 | 2.98 | 3.05 | 90.7 | 93.3 |
| | S99F, S101L, S103T, | 10 | 3.12 | | | |
| | V104I, A172K, A194P, | | | | | |
| | V205I, Q206L, Y209W, | | | | | |
| | A215K, S259D, N261W, | | | | | |
| | L262E | | | | | |
| 64 | S3T, S9E, N43R, N76D, | 10 | 2.10 | 2.10 | | |
| | S99F, S101L, S103T, | | | | | |
| | V104I, M175A, A194P, | | | | | |
| | V205I, Q206L, Y209W, | | | | | |
| | A215K, S259D, N261W, | | | | | |
| | L262E | | | | | |
| 65 | S3T, S9E, N43R, N76D, | 10 | 2.42 | 2.42 | | |
| | S99F, S101L, S103T, | | | | | |
| | V104I, M175Y, A194P, | | | | | |
| | V205I, Q206L, Y209W, | | | | | |
| | A215K, S259D, N261W, | | | | | |
| | L262E | | | | | |
| 66 | S3T, S9E, N43R, N76D, | 5 | 2.92 | 2.89 | 91.1 | 93.3 |
| | S99F, S101L, S103T, | 10 | | | | |
| | V104I, A194P, V205I, | | | | | |
| | Q206L, Y209W, A215K, | | | | | |
| | S259D, N261W, L262E | | | | | |

### Example 4. Screening of First-Generation Protease Variants Based on SEQ ID NO:3

20 different protease variants based on SEQ ID NO:3 were prepared and purified according to the above described procedure and tested in the above described turbidity assay. Compared to SEQ ID NO:3, these variants contain the following substitutions:

| SEQ ID NO: 1 | SEQ ID NO: 3 | SEQ ID NO: 4 |
|---|---|---|
| (BPN′ numbering, WO | (BPN′ numbering, | (BPN′ numbering, |
| 1989/06279) | Needleman-Wunsch) | Needleman-Wunsch) |
| S3 (S3T) | T3 (N/A) | T3 (N/A) |
| S9 (S9E) | P9 (P9E) | T9 (T9E) |
| N43 (N43R) | N43 (N43R) | T43 (T43R) |
| N76 (N76D) | D76 (N/A) | N76 (N76D) |
| G97 (G97D) | N97 (N97D) | G97 (G97D) |
| S99 (S99F) | S99 (S99F) | N99 (N98F) |
| S101 (S101L) | S101 (S101L) | R101 (R101L) |
| S103 (S103T) | S103 (S103T) | S103 (S102T) |
| V104 (V104I) | Y104 (Y104I) | V104 (V104I) |
| G127 (G127I) | G127 (G127I) | G127 (G127I) |
| S161 (S161W/R/Y/L/V) | N161 (N161W/Y/L/V) | N/A |
| S163 (S163R) | N163 (N163R) | G163 (G163R) |
| A172 (A172K) | D172 (D171K) | A172 (A172K) |
| A174 (A174G/V) | V174 (V174G) | A174 (A174G/V) |
| M175 (M175A/Y/D/T/V/I) | I175 (I175A/Y/D/T/V) | M175 (M175A/Y/D/T/V/I |
| A176 (A176S) | A176 (A176S) | A176 (A176S) |
| A194 (A194P) | A194 (A194P) | T194 (T194P) |
| V205 (V205I) | V205 (V205I) | I205 (N/A) |
| Q206 (Q206L) | Y206 (Y206L) | Q206 (Q206L) |
| Y209 (Y209W) | Y209 (Y209W) | Y209 (Y209W) |
| A215 (A215K) | A215 (A215K) | A215 (A215K) |
| S259 (S259D) | P259 (P259D) | N259 (N259D) |
| N261 (N261W) | F261 (F261W) | S261 (S261W) |
| L262 (L262E) | Y262 (Y262E) | Q262 (Q262E) |

| SEQ ID NO: | Substitutions vs. SEQ ID NO: 3 (using BPN′ numbering) |
|---|---|
| 67 | S99F, S101L |
| 68 | S99F, S101L, A194P |
| 69 | S99F S101L A215K |
| 70 | S99F, S101L, S103T, Y104I |
| 71 | S99F, S101L, S156E, N163R |
| 72 | S99F, S101L, S103T, Y104I, A194P |
| 73 | S99F, S101L, S156E, N163R, A194P |
| 74 | S99F, S101L, A194P, A215K |
| 75 | S99F, S101L, S156E, N163R, A215K |
| 76 | S99F, S101L, S103T, Y104I, S156E, N163R, A194P |
| 77 | S99F, S101L, S103T, Y104I, A194P, A215K |
| 78 | S99F, S101L, S103T, Y104I, S156E, N163R, A215K |
| 79 | S99F, S101L, S103T, Y104I, S156E, N163R, A194P, |
| | A215K |
| 80 | S99F, S101L, N163R |
| 81 | S99F, S101L, S103T, Y104I, N163R |
| 82 | S99F, S101L, S103T, Y104I, N163R, A194P, A215K |
| 83 | S99F, S101L, S103T, Y104I, A215K |
| 84 | S99F, S101L, S156E, N163R, A194P, A215K |
| 85 | S99F, S101L, S103T, Y104I, N163R, A215K |
| 86 | S99F, S101L, S103T, Y104I, N163R, A194P |

All variants were dosed at same level of 50 μg enzyme protein, allowing ΔT(t) to be compared. The starting turbidity (Tₛₜ) of the PLA emulsion was approx. 500 NTU, and thus the maximal ΔT(t), which would be equal to a completely clear solution (0 NTU), is consequently 500 NTU.

The polyester degrading effect of the SEQ ID NO:3 was very low, as evidenced by a flattened curve similar to SEQ ID NO:5 in FIG. 2 (data not shown), whereas the variants showed polyester degrading activity over the entire course of the experiment (50 min of recorded reaction time). Consequently, it is not feasible to calculate an improvement factor compared to SEQ ID NO:3, since this factor will be biased by the time frame selected for calculating ΔT(t) of SEQ ID NO:3. However, by comparing ΔT(50 min) values, an approximated improvement factor can be estimated. Variants defined as SEQ ID NOs:68-87 all showed improved polyester degrading activity compared to SEQ ID NO:3, with approximated improvement factors in the range from 6 to 14 (corresponding to 6× and 14× improvements, respectively).

ΔT(t) at different time points

| | ΔT(0 | ΔT(10 | ΔT(20 | ΔT(30 | ΔT(40 | ΔT(50 |
|---|---|---|---|---|---|---|
| SEQ ID NO: | min) | min) | min) | min) | min) | min) |
| 3 | 0 | 23 | 27 | 28 | 25 | 35 |
| 67 | 0 | 113 | 200 | 267 | 311 | 351 |
| 68 | 0 | 110 | 214 | 276 | 333 | 369 |
| 69 | 0 | 291 | 422 | 461 | 476 | 484 |
| 70 | 0 | 109 | 195 | 252 | 310 | 352 |
| 71 | 0 | 56 | 90 | 140 | 180 | 213 |
| 72 | 0 | 100 | 192 | 260 | 298 | 340 |
| 73 | 0 | 63 | 107 | 157 | 194 | 232 |
| 74 | 0 | 290 | 423 | 469 | 485 | 492 |
| 75 | 0 | 148 | 265 | 338 | 397 | 435 |
| 76 | 0 | 70 | 118 | 171 | 207 | 245 |
| 77 | 0 | 269 | 396 | 449 | 471 | 481 |
| 78 | 0 | 162 | 273 | 363 | 409 | 442 |
| 79 | 0 | 139 | 242 | 322 | 377 | 413 |
| 80 | 0 | 373 | 452 | 467 | 475 | 478 |
| 81 | 0 | 402 | 458 | 471 | 476 | 479 |
| 82 | 0 | 362 | 484 | 489 | 491 | 492 |
| 83 | 0 | 269 | 397 | 451 | 472 | 481 |
| 84 | 0 | 163 | 272 | 357 | 413 | 443 |
| 85 | 0 | 483 | 494 | 497 | 498 | 499 |
| 86 | 0 | 420 | 475 | 486 | 492 | 494 |

### Example 5. Screening of First-Generation Protease Variants Based on SEQ ID NO:4

Five different protease variants based on SEQ ID NO:4 were prepared and purified according to the above described procedure and tested in the above described turbidity assay. Compared to SEQ ID NO:4, these variants contain the following substitutions:

| SEQ ID NO: | Substitutions vs. SEQ ID NO: 4 (using BPN′ numbering) |
|---|---|
| 87 | N99F, R101L, T194P, A215K |
| 88 | N99F, R101L, S103T, V104I |
| 89 | N99F, R101L, S103T, V104I, T194P |
| 90 | N99F, R101L, S103T, V104I, A215K |
| 91 | N99F, R101L, S103T, V104I, N156E, G163R, T194P |

All variants were dosed at same level of 50 μg enzyme protein, allowing ΔT(t) to be compared. The starting turbidity (Tₛₜ) of the PLA emulsion was approx. 500 NTU, and thus the maximal ΔT(t), which would be equal to a completely clear solution (0 NTU), is consequently 500 NTU.

The polyester degrading effect of the SEQ ID NO:4 was very low, as evidenced by a flattened curve similar to SEQ ID NO:5 in FIG. 2 (data not shown), whereas the variants showed polyester degrading activity over the entire course of the experiment (40 min of recorded reaction time). Consequently, it is not feasible to calculate an improvement factor compared to SEQ ID NO:4, since this factor will be biased by the time frame selected for calculating ΔT(t) of SEQ ID NO:4. However, by comparing ΔT(40 min) values, an approximated improvement factor can be estimated. Variants defined as SEQ ID NOs:87-91 all showed improved polyester degrading activity compared to SEQ ID NO:4, with approximated improvement factors in the range from 4 to 8 (corresponding to 4× and 8× improvements, respectively).

ΔT(t) at different time points

| SEQ | ΔT(0 | ΔT(10 | ΔT(20 | ΔT(30 | ΔT(40 |
|---|---|---|---|---|---|
| ID NO: | min) | min) | min) | min) | min) |
| 4 | 0 | 23 | 22 | 25 | 30 |
| 87 | 0 | 36 | 84 | 111 | 162 |
| 88 | 0 | 47 | 68 | 89 | 113 |
| 89 | 0 | 38 | 64 | 90 | 114 |
| 90 | 0 | 78 | 147 | 203 | 255 |
| 91 | 0 | 73 | 143 | 198 | 244 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A protease variant having a sequence identity of at least 60%, but less than 100%, to SEQ ID NO:1; wherein the variant comprises the substitutions X99F and X101L; wherein the variant further comprises at least one substitution selected from the group consisting of X3T, X97D, X103T, X104I, X127I, X161W, X161R, X161Y, X161L, X161V, X163R, X172K, X174G, X174V, X175A, X175Y, X175D, X175T, X175V, X175I, X176S, X194P, and X215K; wherein the variant has polyester degrading activity and, optionally, protease activity; and wherein position numbers are based on the numbering of SEQ ID NO:2.

2. The variant according to claim 1, wherein the variant comprises at least one substitution selected from the group consisting of X103T, X104I, X127I, X194P, and X215K.

3. The variant according to claim 1, which comprises the substitutions
a) X99F, X101L, and X103T;
b) X99F, X101L, and X104I;
c) X99F, X101L, and X127I;
d) X99F, X101L, and X194P;
e) X99F, X101L, and X215K;
f) X99F, X101L, X103T, and X104I;
g) X99F, X101L, X103T, X104I, and X194P;
h) X99F, X101L, X103T, X104I, and X215K;
i) X99F, X101L, X103T, X104I, X194P, and X215K; or
j) X99F, X101L, X103T, X104I, X127I, X194P, and X215K.

4. The variant according to claim 1, which further comprises at least three substitutions selected from the group consisting of X9E, X43R, X76D, X205I, X206L, X209W, X259D, X261W, and X262E.

5. The variant according to claim 4, which comprises the substitutions:
a) X9E, X43R, X76D, X99F, X101L, X205I, X206L, X209W, X215L, X259D, X261W, and X262E;
b) X9E, X43R, X76D, X99F, X101L, X127I, X205I, X206L, X209W, X259D, X261W, and X262E;
c) X9E, X43R, X76D, X99F, X101L, X127I, X205I, X206L, X209W, X215K, X259D, X261W, and X262E;
d) X9E, X43R, X76D, X99F, X101L, X103T, X127I, X205I, X206L, X209W, X215K, X259D, X261W, and X262E;
e) X9E, X43R, X76D, X99F, X101L, X104I, X127I, X205I, X206L, X209W, X215K, X259D, X261W, and X262E;
f) X9E, X43R, X76D, X99F, X101L, X103T, X104I, X205I, X206L, X209W, X215K, X259D, X261W, and X262E
g) X9E, X43R, X76D, X99F, X101L, X103T, X104I, X127I, X205I, X206L, X209W, X215K, X259D, X261W, and X262E; or
h) X9E, X43R, X76D, X99F, X101L, X103T, X104I, X127I, X194P, X205I, X206L, X209W, X215K, X259D, X261W, and X262E.

6. The variant according to claim 1, which further comprises at least one, substitutions selected from the group consisting of X97D, X172K, X174G, X174V, and X176S.

7. The variant according to claim 1, which further comprises a substitution selected from the group consisting of X161W, X161R, X161Y, X161L, and X161V, and/or a substitution selected from the group consisting of X175A, X175Y, X175D, X175T, X175V, and X175I.

8. The variant according to claim 1, which has on par or improved polyester degrading activity, preferably PLA degrading activity.

9. The variant according to claim 1, which has on par or improved thermostability and/or on par or improved proteolytic stability.

10. The variant according to claim 1, which has on par or improved polyester specificity, preferably PLA specificity.

11. An isolated polynucleotide encoding a variant of claim 1.

12. A nucleic acid construct or expression vector comprising or a recombinant host cell transformed with the polynucleotide of claim 11.

13. A composition comprising a protease variant according to claim 1.

14. A method of producing a protease variant according to claim 1, the method comprising:
a) cultivating a recombinant host cell transformed with a polynucleotide encoding a variant of claim 1 under conditions suitable for expression of the variant; and
b) recovering the variant.

15. A method of degrading a polyester containing material, the method comprising:
a) contacting the polyester containing material with a variant according to claim 1; and, optionally
b) recovering the resulting monomers and/or oligomers.

16. The method of claim 15, wherein the polyester containing material comprises at least one polyester selected from polylactic acid (PLA), polytrimethylene terephthalate (PTT), polybutylene terephthalate (PBT), polyethylene isosorbide terephthalate (PEIT), polyethylene terephthalate (PET) polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly(ethylene adipate) (PEA) and blends/mixtures of these materials; preferably the at least one polyester comprises or consists of polylactic acid (PLA).

17. A plastic material or product comprising (a) a protease variant according to claim 1; and (b) at least one polyester; preferably PLA.

18. A masterbatch composition comprising (a) a protease variant according to claim 1; and (b) at least one polyester, preferably PLA.

19. A method for producing a plastic material or product of claim 17, the method comprising:
a) providing a variant of claim 1; and
b) mixing the variant with at least one polyester at a temperature at which the polyester is in a partially or totally molten state, preferably by extrusion.

20. (canceled)

21. The method of claim 19 wherein the at least one polyester comprises or consists of PLA.

22. A method of degrading a polyester containing material, the method comprising contacting the polyester material with a variant of claim 1.


SEQUENCE LISTING (91 sequences)

SEQ ID NO: 1 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 274 aa, Unknown
  Preferenz P300
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSSGSGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 4 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 5 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 6 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 7 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSIRYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 8 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNEGAGSIRYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 9 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGSVSSIAQGLEWAGNNGMHVANLSLGSPSPSATLEQAVNSATSRGVLVVAASGNEGAGSIRYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 10 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGSISSIAQGLEWAGNNGMHVANLSLGSPSPSATLEQAVNSATSRGVLVVAASGNEGAGSIRYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 11 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGLGSISSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR

SEQ ID NO: 12 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYKSLNGTSMATPHVAGAAALVKQKNPSWSNVQIRNHLKNTATSLGDTWEYGSGLVNAEAATR

SEQ ID NO: 13 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 14 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSIRYPARYANAMAVGATDQNNNRASFSQYGAGLDIVAPGVNILSTWPGSTYKSLNGTSMATPHVAGAAALVKQKNPSWSNVQIRNHLKNTATSLGDTWEYGSGLVNAEAATR

SEQ ID NO: 15 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLISPSPSATLEQAVNSATSRGVLVVAASGNSGAGSISYPARYANAMAVGATDQNNNRASFSQYGAGLDIVAPGVNILSTWPGSTYKSLNGTSMATPHVAGAAALVKQKNPSWSNVQIRNHLKNTATSLGDTWEYGSGLVNAEAATR

SEQ ID NO: 16 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLISPSPSATLEQAVNSATSRGVLVVAASGNSGAGSISYPARYANAMAVGATDQNNNRASFSQYGAGLDIVAPGVNILSTWPGSTYKSLNGTSMATPHVAGAAALVKQKNPSWSNVQIRNHLKNTATSLGDTWEYGSGLVNAEAATR

SEQ ID NO: 17 - protein, 269 aa, Artificial Sequence
  Variant
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLISPSPSATLEQAVNSATSRGVLVVAASGNSGAGSISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 18 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLISPSPSATLEQAVNSATSRGVLVVAASGNSGAGSISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 19 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANGMSVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 20 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNGMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 21 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGWISYPARYANAAAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 22 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANAAAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 23 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAASVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 24 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNAAAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 25 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGYISYPARYANAYAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 26 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGLISYPARYANAYAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 27 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAYSVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 28 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANVYAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 29 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANADAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 30 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNADAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 31 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGWISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 32 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGVISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 33 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGYISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 34 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 35 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGLISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 36 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANATSVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 37 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANGTAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 38 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 39 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGWISYPARYANAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 40 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGYISYPARYANAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 41 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 42 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGLISYPARYANAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 43 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAVSVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 44 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANGVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 45 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 46 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGWISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 47 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGVISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 48 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGYISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 49 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 50 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGLISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 51 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANGIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 52 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 53 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSIRYPARYKNAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 54 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGWISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 55 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGVISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 56 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGYISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 57 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGRISYPARYANAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 58 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANATAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 59 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAVAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 60 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAIAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 61 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAMSVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 62 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANGMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 63 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYKNAMAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 64 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAAAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 65 - protein, 269 aa, Artificial Sequence
  Variant
AQTVPWGIERVQAPAAHNRGLTGSGVKVAVLDTGISTHPDLRIRGGASFVPGEPSTQDGNGHGTHVAGTIAALDNSIGVLGVAPSAELYAVKVLGAFGLGTISSIAQGLEWAGNNGMHVANLSLGSPSPSATLEQAVNSATSRGVLVVAASGNSGAGSISYPARYANAYAVGATDQNNNRASFSQYGPGLDIVAPGVNILSTWPGSTYKSLNGTSMATPHVAGAAALVKQKNPSWSNVQIRNHLKNTATSLGDTWEYGSGLVNAEAATR

SEQ ID NO: 66 - protein, 269 aa, Artificial Sequence; identical to SEQ ID NO: 13
  Variant

SEQ ID NO: 67 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 68 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNSGSSGNTNTIQYPAKYDSVIAVGAVDSNSQRASFSSVGPELEVMAPGVGVYSTYPTNTYATLSGTSMASPHVAGAAALILSKHPNLSASQVRNRLSSTATYLGPSFYYGKGLINVEAAAQ

SEQ ID NO: 69 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYKTLSGTSMASPHVAGAAALILSKHPNLSASQVRNRLSSTATYLGPSFYYGKGLINVEAAAQ

SEQ ID NO: 70 - protein, 274 aa, Artificial Sequence; identical to SEQ ID NO: 69
  Variant

SEQ ID NO: 71 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNEGSSGNTNTIRYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 72 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIQYPAKYDSVIAVGAVDSNSQRASFSSVGPELEVMAPGVGVYSTYPTNTYATLSGTSMASPHVAGAAALILSKHPNLSASQVRNRLSSTATYLGPSFYYGKGLINVEAAAQ

SEQ ID NO: 73 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNEGSSGNTNTIRYPAKYDSVIAVGAVDSNSQRASFSSVGPELEVMAPGVGVYSTYPTNTYATLSGTSMASPHVAGAAALILSKHPNLSASQVRNRLSSTATYLGPSFYYGKGLINVEAAAQ

SEQ ID NO: 74 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNSGSSGNTNTIQYPAKYDSVIAVGAVDSNSQRASFSSVGPELEVMAPGVGVYSTYPTNTYKTLSGTSMASPHVAGAAALILSKHPNLSASQVRNRLSSTATYLGPSFYYGKGLINVEAAAQ

SEQ ID NO: 75 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNEGSSGNTNTIRYPAKYDSVIAVGAVDSNSQRASFSSVGAELEVMAPGVGVYSTYPTNTYKTLSGTSMASPHVAGAAALILSKHPNLSASQVRNRLSSTATYLGPSFYYGKGLINVEAAAQ

SEQ ID NO: 76 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNEGSSGNTNTIRYPAKYDSVIAVGAVDSNSQRASFSSVGPELEVMAPGVGVYSTYPTNTYATLSGTSMASPHVAGAAALILSKHPNLSASQVRNRLSSTATYLGPSFYYGKGLINVEAAAQ

SEQ ID NO: 77 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIQYPAKYDSVIAVGAVDSNSQRASFSSVGPELEVMAPGVGVYSTYPTNTYKTLSGTSMASPHVAGAAALILSKHPNLSASQVRNRLSSTATYLGPSFYYGKGLINVEAAAQ

SEQ ID NO: 78 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNEGSSGNTNTIRYPAKYDSVIAVGAVDSNSQRASFSSVGAELEVMAPGVGVYSTYPTNTYKTLSGTSMASPHVAGAAALILSKHPNLSASQVRNRLSSTATYLGPSFYYGKGLINVEAAAQ

SEQ ID NO: 79 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNEGSSGNTNTIRYPAKYDSVIAVGAVDSNSQRASFSSVGPELEVMAPGVGVYSTYPTNTYKTLSGTSMASPHVAGAAALILSKHPNLSASQVRNRLSSTATYLGPSFYYGKGLINVEAAAQ

SEQ ID NO: 80 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNSGSSGNTNTIRYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 81 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIRYPAKYDSVIAVGAVDSNSQRASFSSVGAELEVMAPGVGVYSTYPTNTYATLSGTSMASPHVAGAAALILSKHPNLSASQVRNRLSSTATYLGPSFYYGKGLINVEAAAQ

SEQ ID NO: 82 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIRYPAKYDSVIAVGAVDSNSQRASFSSVGPELEVMAPGVGVYSTYPTNTYKTLSGTSMASPHVAGAAALILSKHPNLSASQVRNRLSSTATYLGPSFYYGKGLINVEAAAQ

SEQ ID NO: 83 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIQYPAKYDSVIAVGAVDSNSQRASFSSVGAELEVMAPGVGVYSTYPTNTYKTLSGTSMASPHVAGAAALILSKHPNLSASQVRNRLSSTATYLGPSFYYGKGLINVEAAAQ

SEQ ID NO: 84 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGSYSGIVSGIEWATTNGMDVINMSLGSPSGSTAMKQAVDNAYARGVVVVAAAGNEGSSGNTNTIRYPAKYDSVIAVGAVDSNSQRASFSSVGPELEVMAPGVGVYSTYPTNTYKTLSGTSMASPHVAGAAALILSKHPNLSASQVRNRLSSTATYLGPSFYYGKGLINVEAAAQ

SEQ ID NO: 85 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIRYPAKYDSVIAVGAVDSNSQRASFSSVGAELEVMAPGVGVYSTYPTNTYKTLSGTSMASPHVAGAAALILSKHPNLSASQVRNRLSSTATYLGPSFYYGKGLINVEAAAQ

SEQ ID NO: 86 - protein, 274 aa, Artificial Sequence
  Variant
AQTVPYGIPLIKADKVQAQGFKGANVKVAVLDTGIQASHPDLNVVGGASFVAGEAYNTDGNGHGTHVSGTVAALDNNIGVLGVAPSVSLYAVKVLNSFGLGTISGIVSGIEWATTNGMDVINMSLGSPSGSTAMKQAVDNAYARGVVVVAAAGNSGSSGNTNTIRYPAKYDSVIAVGAVDSNSQRASFSSVGPELEVMAPGVGVYSTYPTNTYATLSGTSMASPHVAGAAALILSKHPNLSASQVRNRLSSTATYLGPSFYYGKGLINVEAAAQ

SEQ ID NO: 87 - protein, 269 aa, Artificial Sequence
  Variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGAFGLGSVSGIAQGLEWAATNNMHIANMSLGSDAPSTTLERAVNYATSRGVLVIAATGNNGTGSIGYPARYANAMAVGATDQNNRRASFSQYGPGIDIVAPGVGIQSTYLNNSYKSLDGTSMATPHVAGVAALVKQKNPSWNATQIRNHLKNTATNLGNSSQFGSGLVNADAATR

SEQ ID NO: 88 - protein, 269 aa, Artificial Sequence
  Variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGAFGLGTISGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 89 - protein, 269 aa, Artificial Sequence
  Variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGAFGLGTISGIAQGLEWAATNNMHIANMSLGSDAPSTTLERAVNYATSRGVLVIAATGNNGTGSIGYPARYANAMAVGATDQNNRRASFSQYGPGIDIVAPGVGIQSTYLNNSYASLDGTSMATPHVAGVAALVKQKNPSWNATQIRNHLKNTATNLGNSSQFGSGLVNADAATR

SEQ ID NO: 90 - protein, 269 aa, Artificial Sequence
  Variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGAFGLGTISGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYKSLDGTSMATPHVAGVAALVKQKNPSWNATQIRNHLKNTATNLGNSSQFGSGLVNADAATR

SEQ ID NO: 91 - protein, 269 aa, Artificial Sequence
  Variant
QQTVPWGITRVQAPTVHNRGITGSGVKVAILDTGIAQHSDLTIRGGASFVPGESTTADLNGHGTHVAGTVAALNNSIGVIGVAPSADLYAVKVLGAFGLGTISGIAQGLEWAATNNMHIANMSLGSDAPSTTLERAVNYATSRGVLVIAATGNEGTGSIRYPARYANAMAVGATDQNNRRASFSQYGPGIDIVAPGVGIQSTYLNNSYASLDGTSMATPHVAGVAALVKQKNPSWNATQIRNHLKNTATNLGNSSQFGSGLVNADAATR